(19) United States
(12) Patent Application Publication
(10) Pub. No.: US 2025/0171407 A1
(43) Pub. Date: May 29, 2025

(54) ULTRAVIOLET ABSORBER

(71) Applicant: BASF SE, Ludwigshafen am Rhein (DE)

(72) Inventors: Barbara WINKLER, Schweizerhalle (Muttenz) (CH); Delphine KIMPEL, Ludwigshafen am Rhein (DE)

(21) Appl. No.: 18/840,156

(22) PCT Filed: Feb. 8, 2023
(86) PCT No.: PCT/EP2023/053065

§ 371 Date: Aug. 21, 2024

(30) Foreign Application Priority Data
Feb. 22, 2022 (EP) 22157855.2
Mar. 11, 2022 (EP) 22161493.6

(51) Int. Cl. C07D 249/20; C07D 251/24; C08K 5/3475; C08K 5/3492; C09D 5/32

(52) U.S. Cl. CPC C07D 249/20; C07D 251/24; C09D 5/32; C08K 5/3475; C08K 5/3492

(57) ABSTRACT

The presently claimed invention relates to an ultraviolet absorber compound and a process for the preparation thereof.

### FIELD OF THE INVENTION

The presently claimed invention relates to an ultraviolet absorber compound and a process for the preparation thereof.

### BACKGROUND OF THE INVENTION

UV absorbers are an important class of organic compounds which have a wide variety of applications. One of the most important areas of application is the protection and stabilization of organic materials such as plastics, polymers, coating materials, and photographic recording materials against damages by light, heat, oxygen, or environmental forces. Other areas of application include cosmetics, fibres, dyes, etc.

The triazine and benzotriazole based compounds are important classes of UV absorbers. The UV absorbers of presently claimed invention is prepared either by esterification, amidation from corresponding carboxylic acid or esters by tans esterification of the corresponding triazine and benzotriazole compounds. The esterification and trans esterification methods are well known in the literature.

WO 2019/192982 A1 describes the formation of benzotriazole polyglycerols by the transesterification reaction methyl 3-[3-(benzotriazol-2-yl)-5-tert-butyl-4-hydroxy-phenyl]propanoate with polyglycerol-14 mediated by the tin catalyst tin-(II)-2-ethylhexanoate. The reaction mixture was stirred at 185-195° C. for 64 hours for completion.

WO 02/098968 A1 discloses the production of Tinuvin® 384 by performing a transesterification reaction of methyl 3-[3-(benzotriazol-2-yl)-5-tert-butyl-4-hydroxy-phenyl]propanoate with isooctanol in the presence of dibutyltin oxide at 175° C. at reduced pressure. After the completion of the reaction the product is separated from the catalyst by distillation.

WO98/03489 A1 and WO2001056998 A1 describe the formation of polyoxy alkylene substituted benzotriazole esters and amides which are formed by the reaction methyl 3-[3-(benzotriazol-2-yl)-5-tert-butyl-4-hydroxy-phenyl]propanoate with diamines such as Jeffamines® or polyethylene monoalcohol using lithium amide as catalyst.

US 2012/0059095 A1 discloses high-molecular benzotriazole UV absorbers are produced via esterification reaction starting from 2-(2′-hydroxy-5′-hydroxypropylphenyl)-2H-benzotriazole and an alcohol in the presence of methanesulfonic acid. The reaction is carried out at 165° C. for 24 hours.

WO 2010115721 A2 and WO2010103021 A1 teach the formation of benzotriazole esters by a transesterification reaction of methyl 3-[3-(benzotriazol-2-yl)-5-tert-butyl-4-hydroxy-phenyl]propanoate with polyethylene glycols in the presence of titanium(IV) isopropoxide. The reaction mixtures were stirred at 155° C. for 24-52 hours for completion.

WO2004033699 A1 teaches the production of benzotriazole esters via transesterification of methyl 3-[3-(benzotriazol-2-yl)-5-tert-butyl-4-hydroxy-phenyl]propanoate with 1-n-octanol by using enzymatic catalysis. Besides the use of a high excess of alcohol the reaction takes 2 to 5 days.

The prior art processes have the disadvantages of using drastic reaction conditions such as high reaction temperature, environmentally hazardous catalyst, and longer reaction time. The environmentally hazardous catalyst such as organotin compounds are under regulatory pressure due to toxicity concerns. With the regulatory concerns adhered to specific tin compounds those compounds are in general avoided by many producers and formulators. In some cases, such catalysts are tediously removed after reaction, for example, by distillation of the desired product or using an adsorbent for catalyst. Some catalysts such as titanates are known to be deactivated by the water sometimes present in raw materials such as alcohols, amines or esters which require anhydrous reaction conditions or additional drying steps.

Thus, the object of the presently claimed invention is to provide UV absorber compounds having environmentally benign residual catalyst having similar or better UV absorption as in prior art, and process with reduced reaction temperature, shorter reaction time, and of course use of catalyst which is benign to environment.

### SUMMARY OF THE INVENTION

Surprisingly, it was found that the esterification, amidation or transesterification of the carboxylic acid or esters in the presence of at least one catalyst selected from Zr compound, Bi compound, Zn compound, or a combination of two or more thereof can reduce the reaction time and/or reduce the reaction temperature. The product thus formed display similar or better UV absorption even in the presence of residual Zr compound, Bi compound, Zn compound, or a combination or free metal forms.

Accordingly, in first aspect of the presently claimed invention is directed to a process for obtaining an ester or an amide of a compound of formula (I) comprising at least the step of:
a) providing at least one compound of formula (I),

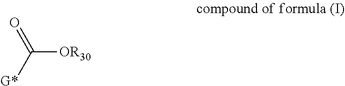

wherein
G* is selected from formula (A), or formula (B)

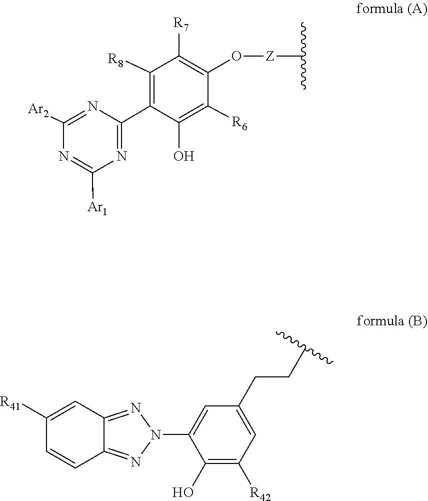

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C), or a moiety of formula (M),

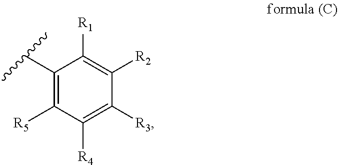

wherein R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, OR,
R selected from hydrogen, substituted or unsubstituted, linear or branched C₁-C₂₄ alkyl,

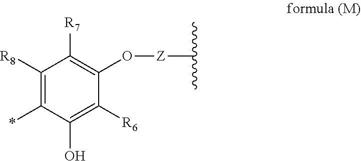

wherein the dotted line is a single bond between formula (M) and the triazinyl ring of formula (A), Z is as defined as above,
   R₆, R₇ and R₈ are independently of each other selected from hydrogen, substituted or unsubstituted, linear or branched C₁-C₂₄ alkyl,
   R₄₁, and R₄₂ independently of each other, are selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
   R₃₀ is selected from hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
b) providing at least one alcohol (D) or an amine (E) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.

The second aspect of the presently claimed invention is directed to an ester or an amide of a compound of formula (I) obtained according first aspect, wherein the ester or the amide of the compound of formula (I) comprises Zr, Zn, Bi, or combination of two or more in free form or a compound form in an amount in the range of 0.0001 to 5.0 wt. % based on total amount of the ester or the amide of the compound of formula (I).

The third aspect of the presently claimed invention is directed to an ester or an amide of a compound of formula (I) comprising Zr, Zn, Bi, or combination of two or more in free form or a compound form in an amount in the range of 0.0001 to 5.0 wt. % based on total amount of the ester or the amide of the compound of formula (I).

The fourth aspect of the presently claimed invention is directed to a composition comprising an ester or an amide of a compound of formula (I) according second aspect or third aspect.

The fifth aspect of the presently claimed invention is directed to use of an ester or an amide of a compound of formula (I) according to second aspect or third aspect as UV light stabilizer.

The sixth aspect of the presently claimed invention is directed to a method of stabilizing a coating/composition against UV light comprising at least the steps of:
i. proving an ester or an amide of a compound of formula (I) according to second aspect or third aspect or a composition according to fourth aspect.

### DETAILED DESCRIPTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment, but may.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

In a first embodiment, the presently claimed invention is directed to a process for obtaining an ester or an amide of a compound of formula (I) comprising at least the step of:
a) providing at least one compound of formula (I),

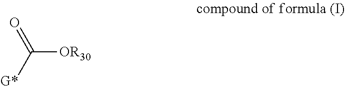

wherein
G* is selected from formula (A), or formula (B)

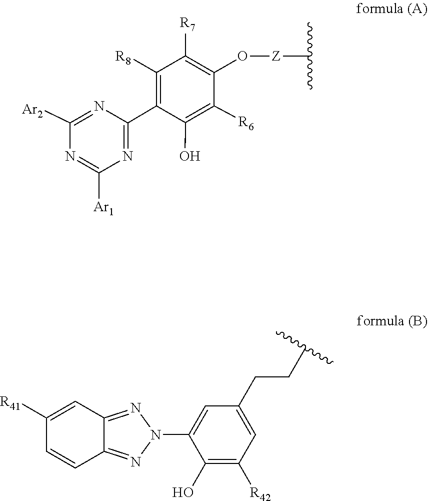

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C), or a moiety of formula (M),

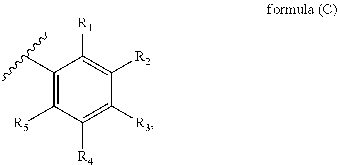

wherein R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, OR,
R selected from hydrogen, substituted or unsubstituted, linear or branched C₁-C₂₄ alkyl,

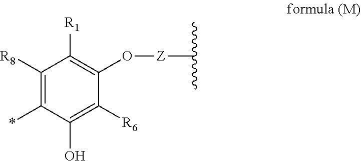

wherein the dotted line is a single bond between formula (M) and the triazinyl ring of formula (A), Z is as defined as above,
R₆, R₇ and R₈ are independently of each other selected from hydrogen, substituted or unsubstituted, linear or branched C₁-C₂₄ alkyl,
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
b) providing at least one alcohol (D) or an amine (E) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.

Preferably, the presently claimed invention is directed to a process for obtaining an ester or an amide of a compound of formula (I) comprising at least the step of:
a) providing at least one compound of formula (I),
b)

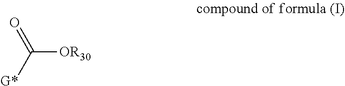

wherein
G* is selected from formula (A), or formula (B)

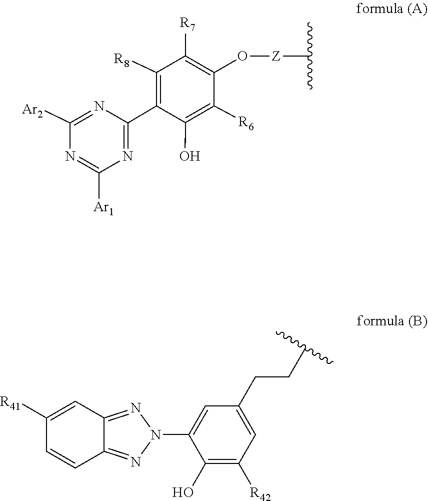

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄alkenylene,
Ar₁ and Ar₂ are independently of each other a moiety of the formula (C), or a moiety of formula (M),

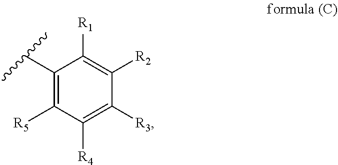

wherein R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, OR,
R selected from hydrogen, substituted or unsubstituted, linear or branched C₁-C₂₄ alkyl,

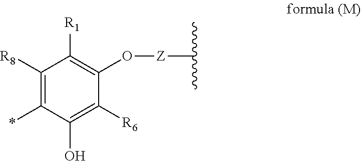

wherein the dotted line is a single bond between formula (M) and the triazinyl ring of formula (A), Z is as defined as above,
R₆, R₇ and R₈ are independently of each other selected from hydrogen, substituted or unsubstituted, linear or branched C₁-C₂₄ alkyl,
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
c) providing at least one alcohol (D) or an amine (E) to obtain a mixture,
d) optionally providing at least one organic acid (F) to mixture obtained in step b) to obtain a reaction mixture, and
e) reacting the mixture obtained in step b) or step c) in the presence of at least one catalyst selected from Zr compound, Bi compound, Zn compound, or a combination of two or more thereof.

More preferably, the presently claimed invention is directed to a process for obtaining an ester or an amide of a compound of formula (I) comprising at least the step of:
a) providing at least one compound of formula (I),

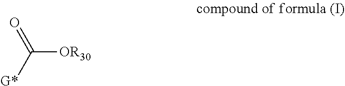

wherein
G* is selected from formula (A), or formula (B)

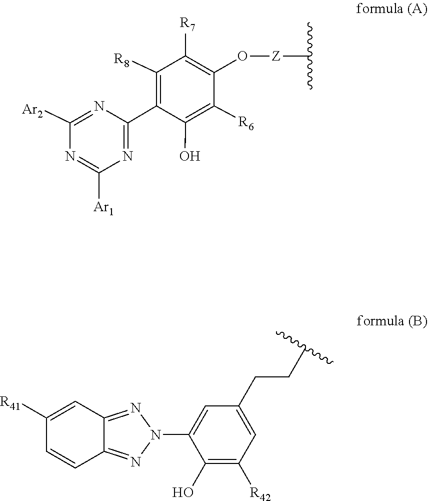

wherein Z is selected from substituted or unsubstituted, linear or branched C₁-C₃₀ alkylene, substituted or unsubstituted, linear or branched 2- to 30-membered hetero alkylene,
Ar₁ and Ar₂ are independently of each other a moiety of the formula (C), or a moiety of formula (M),

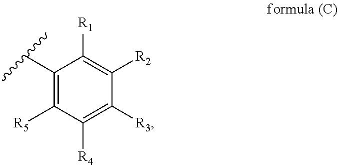

wherein R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, OR,
R selected from hydrogen, substituted or unsubstituted, linear or branched C₁-C₂₄ alkyl,

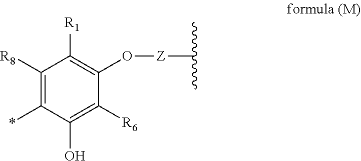

wherein the dotted line is a single bond between formula (M) and the triazinyl ring of formula (A), Z is as defined as above,
R₆, R₇ and R₈ are independently of each other selected from hydrogen, substituted or unsubstituted, linear or branched C₁-C₂₄ alkyl,
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄alkenyl, substituted or unsubstituted C₆-C₂₄ aryl, or substituted or unsubstituted C₇-C₂₄ arylalkyl,
b) providing at least one alcohol (D) or an amine (E) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.

Most preferably, the presently claimed invention is directed to a process for obtaining an ester or an amide of a compound of formula (I) comprising at least the step of:
a) providing at least one compound of formula (I),

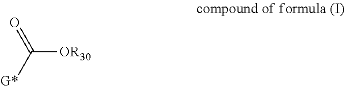

wherein
G* is a formula (B)

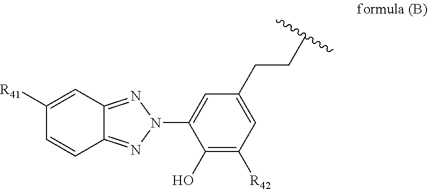

R₄₁, and R₄₂ independently of each other, are selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₁-C₂₄ arylalkyl,
R₃₀ is selected from hydrogen, substituted or unsubstituted, linear or branched C₁-C₂₄ alkyl, substituted or unsubstituted, linear or branched C₂-C₂₄alkenyl, substituted or unsubstituted C₆-C₂₄ aryl, or substituted or unsubstituted C₇-C₂₄ arylalkyl,
b) providing at least one alcohol (D) or an amine (E) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.

In particular, the presently claimed invention is directed to a process for obtaining an ester or an amide of a compound of formula (I) comprising at least the step of:
a) providing at least one compound of formula (I),

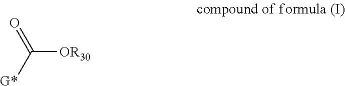

wherein
G* is a formula (B)

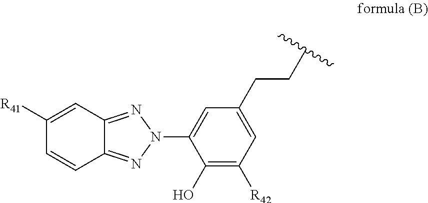

R₄₁, and R₄₂ independently of each other, are selected from hydrogen, halogen, substituted or unsubstituted, linear or branched C₁-C₂₄ alkyl, substituted or unsubstituted C₆-C₂₄ aryl,
R₃₀ is selected from hydrogen, or substituted or unsubstituted, linear or branched C₁-C₆ alkyl,
b) providing at least one alcohol (D) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.

In another preferred embodiment, according to presently claimed invention the —OR₃₀ of compound of formula (I) is different from the alcohol (D).

In another preferred embodiment, the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), 6-methylheptyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof, more preferably the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof, most preferably the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, or a combination of two or more thereof, and in particular, according to presently claimed invention the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

In another preferred embodiment, the alcohol (D) according to presently claimed invention is selected from a monohydric or a polyhydric alcohol.

In another preferred embodiment, the monohydric or the polyhydric alcohol according to presently claimed invention are selected from pentaerythritol, trimethylolpropane, ethylene glycol, substituted or unsubstituted C₁₋₃₀ alkanol having one or more hydroxy functional groups, substituted or unsubstituted C₃₋₃₀ alkenol having one or more hydroxy functional groups, substituted or unsubstituted C₃₋₃₀ heteroalkanol having one or more hydroxy functional groups, substituted or unsubstituted C₄₋₃₀ hetero alkenol having one or more hydroxy functional groups, substituted or unsubstituted aromatic phenol having one or more hydroxy functional groups, substituted or unsubstituted aralkyl having one or more hydroxy functional groups, substituted or unsubstituted cyclic alcohol having one or more hydroxy functional groups, polyalkyleneoxide having one or more hydroxy functional groups, substituted or unsubstituted polyglycerols, or a combination of two or more thereof, wherein the monohydric or the polyhydric alcohol may be substituted or unsubstituted.

In another preferred embodiment, the monohydric or the polyhydric alcohol according to presently claimed invention are selected from pentaerythritol, trimethylolpropane, ethylene glycol, polyethylene glycol, 2-ethyl hexanol, octanol, isooctanol, ditrimethylolethane, ditrimethylolpropane, tristrimethylolpropane, 1,2-propanediol, 1,3-propanediol, diethylene glycol, dipropylene glycol, triethylene glycol, tetraethylene glycol, 1,2-butanediol, 1,4-butanediol, 1,3-butylethylpropanediol, 1,3-methylpropanediol, 1,5-pentanediol, bis(1,4-hydroxymethyl)cyclohexane, glycerol, hexanediol, neopentyl glycol, trimethylolethane, trimethylolpropane, pentaerythritol, bisphenol A, bisphenol B, bisphenol C, bisphenol F, norbornylene glycol, 1,4-benzyldimethanol, 1,4-benzyldiethanol, 2,4-dimethyl-2-ethylhexane-1,3-diol, 1,4-butylene glycol, 2,3-butylene glycol, norbomylene glycol, 1,4-benzyldimethanol, 1,4-benzyldiethanol, 2,4-dimethyl-2-ethylhexane-1,3-diol, 1,4- and 2,3-butylene glycol, di-(3-hydroxyethylbutanediol, 1,5-pentanediol, 1,6-hexanediol, 1,8-octanediol, decanediol, dodecanediol, neopentyl glycol, cyclohexanediol, 3(4),8(9)-bis(hydroxymethyl)-tricyclo[5.2.1.02-6]decane (dicidol), 2,2-bis-(4-hydroxycyclohexyl)propane, 2,2-bis-[4-((3-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris((3-hydroxyethyl) isocyanurate, mannitol, sorbitol, polypropylene glycols, polybutylene glycols, xylylene glycol, neopentyl glycol hydroxypivalate, hydroxyacrylates, polyglycerol, or a combination of two or more thereof, wherein the alcohol (D) may be substituted or unsubstituted having at least one —OH free to react with compound of formula (I), more preferably the monohydric or the polyhydric alcohol according to presently claimed invention are selected from pentaerythritol, trimethylolpropane, ethylene glycol, polyethylene glycol, 2-ethyl hexanol, octanol, isooctanol, ditrimethylolethane, ditrimethylolpropane, tristrimethylolpropane, 1,2-propanediol, 1,3-propanediol, diethylene glycol, dipropylene glycol, triethylene glycol, tetraethylene glycol, 1,2-butanediol, 1,4-butanediol, 1,3-butylethylpropanediol, 1,3-methylpropanediol, 1,5-pentanediol, bis(1,4-hydroxymethyl)cyclohexane, glycerol, hexanediol, neopentyl glycol, trimethylolethane, trimethylolpropane, pentaerythritol, bisphenol A, bisphenol B, bisphenol C, bisphenol F, norbornylene glycol, 1,4-benzyldimethanol, 1,4-benzyldiethanol, 2,4-dimethyl-2-ethylhexane-1,3-diol, 1,4-butylene glycol, 2,3-butylene glycol, norbomylene glycol, 1,4-benzyldimethanol, 1,4-benzyldiethanol, 2,4-dimethyl-2-ethylhexane-1,3-diol, 1,4- and 2,3-butylene glycol, di-(3-hydroxyethylbutanediol, 1,5-pentanediol, 1,6-hexanediol, 1,8-octanediol, decanediol, dodecanediol, neopentyl glycol, cyclohexanediol, 3(4),8(9)-bis(hydroxymethyl)-tricyclo[5.2.1.02-6]decane (dicidol), 2,2-bis-(4-hydroxycyclohexyl)propane, 2,2-bis-[4-((3-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris((3-hydroxyethyl) isocyanurate, mannitol, sorbitol, polypropylene glycols, polybutylene glycols, xylylene glycol, neopentyl glycol hydroxypivalate, hydroxyacrylates, or a combination of two or more thereof, wherein the alcohol (D) may be substituted or unsubstituted having at least one —OH free to react with compound of formula (I).

In another preferred embodiment, the polyhydric alcohol according to presently claimed invention has average number of hydroxy groups in the range of 1 to 100, more preferably the polyhydric alcohol according to presently claimed invention has average number of hydroxy groups in the range of 1 to 80, even more preferably the polyhydric alcohol according to presently claimed invention has average number of hydroxy groups in the range of 1 to 60, most preferably the polyhydric alcohol according to presently claimed invention has average number of hydroxy groups in the range of 2 to 40, and in particular preferably the polyhydric alcohol according to presently claimed invention has average number of hydroxy groups in the range of 1 to 20.

In another preferred embodiment, the polyglycerol according to presently claimed invention is a compound of formula (L),

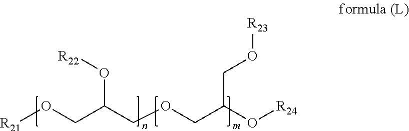

wherein m and n are independently from each other an integer from 0 to 20;
wherein m+n≥2;
wherein R₂₁, R₂₂, R₂₃ and R₂₄ are independently of each other selected from the group consisting of hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, —[(C═O)—(CH₂)ₜ—NH]ⱼ—R₂₅, —[(C═O)—(CH₂)ₜ—O]ⱼ—R₂₅ and —(C═O)R₂₆;
wherein R₂₅ is selected from the group consisting of hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl and substituted or unsubstituted C₇-C₂₄ arylalkyl; “t” is an integer in the range of 4 to 11 and “j” is an integer in the range of 1 to 100; R₂₆ is selected from the group consisting of substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl and substituted or unsubstituted C₇-C₂₄ arylalkyl; with the proviso that at least one of R₂₁, R₂₂, R₂₃ or R₂₄ is hydrogen,
or in case that none of R₂₁, R₂₂, R₂₃ and R₂₄ is hydrogen at least one of R₂₁, R₂₂, R₂₃ or R₂₄ is having at least one substituent selected from —OH, or —NH₂, more preferably the polyglycerol according to presently claimed invention is a compound of formula (L),

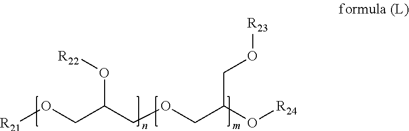

wherein m and n are independently from each other an integer from 0 to 20;
wherein m+n≥2;
wherein R₂₁, R₂₂, R₂₃ and R₂₄ are independently of each other selected from the group consisting of hydrogen, substituted or unsubstituted, linear or branched C₁-C₂₄ alkyl, —[(C═O)—(CH₂)ₜNH]ⱼ—R₂₅, —[(C═O)—(CH₂)ₜ—O]ⱼ—R₂₅ and —(C═O)R₂₆;
wherein R₂₅ is selected from the group consisting of hydrogen, substituted or unsubstituted, linear or branched C₁-C₂₄ alkyl, substituted or unsubstituted, linear or branched C₂-C₂₄alkenyl; “t” is an integer in the range of 5 to 7 and “j” is an integer in the range of 1 to 50; R₂₆ is selected from the group consisting of substituted or unsubstituted, linear or branched C₁-C₂₄ alkyl, substituted or unsubstituted, linear or branched C₂-C₂₄alkenyl with the proviso that at least one of R₂₁, R₂₂, R₂₃ or R₂₄ is hydrogen,
or in case that none of R₂₁, R₂₂, R₂₃ and R₂₄ is hydrogen at least one of R₂₁, R₂₂, R₂₃ or R₂₄ is having at least one substituent selected from —OH, or —NH₂, most preferably the polyglycerol according to presently claimed invention is a compound of formula (L),

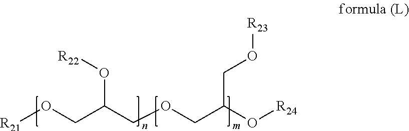

wherein m and n are independently from each other an integer from 0 to 20;
wherein m+n≥2;
wherein R₂₁, R₂₂, R₂₃ and R₂₄ are independently of each other selected from the group consisting of hydrogen, substituted or unsubstituted, linear or branched C₁-C₂₄ alkyl, —[(C═O)—(CH₂)ₜNH]ⱼ—R₂₅, —[(C═O)—(CH₂)ₜ—O]ⱼ—R₂₅ and —(C═O)R₂₆;
wherein R₂₅ is selected from the group consisting of hydrogen, substituted or unsubstituted, linear or branched C₁-C₂₄ alkyl, substituted or unsubstituted, linear or branched C₂-C₂₄alkenyl; “t” is an integer in the range of 5 to 7 and “j” is an integer in the range of 1 to 20; R₂₆ is selected from the group consisting of substituted or unsubstituted, linear or branched C₁-C₂₄ alkyl, substituted or unsubstituted, linear or branched C₂-C₂₄alkenyl;
with the proviso that at least one of R₂₁, R₂₂, R₂₃ or R₂₄ is hydrogen, or in case that none of R₂₁, R₂₂, R₂₃ and R₂₄ is hydrogen at least one of R₂₁, R₂₂, R₂₃ or R₂₄ is having at least one substituent selected from —OH, or —NH₂, and in particular, the polyglycerol according to presently claimed invention is a compound of formula (L),

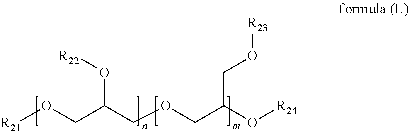

wherein m and n are independently from each other an integer from 0 to 20;
wherein m+n≥2;
wherein R₂₁, R₂₂, R₂₃ and R₂₄ are independently of each other selected from the group consisting of hydrogen, —[(C═O)—(CH₂)ₜ—NH]ⱼ—R₂₅, —[(C═O)—(CH₂)ₜ—O]ⱼ—R₂₅ and —(C═O)R₂₆;
wherein R₂₅ is selected from the group consisting of hydrogen, substituted or unsubstituted, linear or branched C₁-C₂₄ alkyl, and substituted or unsubstituted, linear or branched C₂-C₂₄ alkenyl; “t” is an integer in the range of 5 to 6 and “j” is an integer in the range of 1 to 10; R₂₆ is selected from the group consisting of substituted or unsubstituted, linear or branched C₁-C₂₄ alkyl, and substituted or unsubstituted, linear or branched C₂-C₂₄alkenyl, with the proviso that at least one of R₂₁, R₂₂, R₂₃ or R₂₄ is hydrogen.

In another preferred embodiment, the compound of formula (L) according to presently claimed invention is selected from polyglycerol-3, polyglycerol-4, polyglycerol-6, polyglycerol-10, polyglyceryl-10 stearate, polyglyceryl-10 oleate, polyglyceryl-10 laurate, polyglyceryl-10 dioleate, polyglyceryl-10 distearate, polyglyceryl-10 diisostearate, polyglyceryl-10 dipalmitate, polyglyceryl-2 caprate, polyglyceryl-2 cyprylate, polyglyceryl-2 laurate, polyglyceryl-2 myristate, polyglyceryl-2 isopalmitate, polyglyceryl-2 palmitate, polyglyceryl-2 isostearate, polyglyceryl-2 stearate, polyglyceryl-2 oleate, polyglyceryl-2 isopalmitate/sebacate, polyglyceryl-3 caprate, polyglyceryl-3 laurate, polyglyceryl-3 myristate, polyglyceryl-3 palmitate, polyglyceryl-3 isostearate, polyglyceryl-3 oleate, polyglyceryl-3 stearate, polyglyceryl-3 ricinoleate, polyglyceryl-3 behenate, palm oil polyglyceryl-3 esters, polyglyceryl-3 beeswax, polyglyceryl-3 soyate/shea butterate, sunflower seed oil polyglyceryl-3 esters, polyglyceryl-3 diisostearate, polyglyceryl-3 triisostearate, polyglyceryl-4 caprate, polyglyceryl-4 laurate, polyglyceryl-4 myristate, polyglyceryl-4 palmitate, polyglyceryl-4 isostearate, polyglyceryl-4 oleate, polyglyceryl-4 stearate, polyglyceryl-4 ricinoleate, polyglyceryl-4 behenate, palm oil polyglyceryl-4 esters, polyglyceryl-4 beeswax, polyglyceryl-4 soyate/shea butterate, sunflower seed oil polyglyceryl-4 esters, polyglyceryl-4 dilaurate, polyglyceryl-4 distearate, polyglyceryl-4 tristearate, polyglyceryl-6 caprate, polyglyceryl-6 laurate, polyglyceryl-6 myristate, polyglyceryl-6 palmitate, polyglyceryl-6 isostearate, polyglyceryl-6 oleate, polyglyceryl-6 stearate, polyglyceryl-6 ricinoleate, polyglyceryl-6 behenate, palm oil polyglyceryl-6 esters, polyglyceryl-6 beeswax, polyglyceryl-6 soyate/shea butterate, sunflower seed oil polyglyceryl-6 esters, polyglyceryl-6 dilaurate, polyglyceryl-6 distearate, polyglyceryl-6 tristearate, polyglyceryl-6 dioleate, polyglyceryl-6 tetraoleate, polyglyceryl-6 triisostearate, polyglyceryl-6 pentastearate, polyglyceryl-6 trioleate, polyglyceryl-6 dicarylate, polyglyceryl-6 dicaprate, polyglyceryl-6 tricaprylate, polyglyceryl-10 caprate, polyglyceryl-10 laurate, polyglyceryl-10 myristate, polyglyceryl-10 palmitate, polyglyceryl-10 isostearate, polyglyceryl-10 oleate, polyglyceryl-10 stearate, polyglyceryl-10 ricinoleate, polyglyceryl-10 behenate, palm oil polyglyceryl-10 esters, polyglyceryl-10 beeswax, polyglyceryl-10 soyate/shea butterate, sunflower seed oil polyglyceryl-10 esters, polyglyceryl-10 dilaurate, polyglyceryl-10 distearate, polyglyceryl-10 tristearate, polyglyceryl-10 dioleate, polyglyceryl-10 tetraoleate, polyglyceryl-10 triisostearate, polyglyceryl-10 pentastearate, polyglyceryl-10 trioleate, polyglyceryl-10 dicarylate, polyglyceryl-10 dicaprate, polyglyceryl-10 tricaprylate, polyglyceryl-10 trilaurate, polyglyceryl-10 tetralaurate, polyglyceryl-10 pentahydroxystearate, polyglyceryl-20 hexacaprylate, polyglyceryl-20 heptacaprylate, polyglyceryl-20 octaisononanoate, polyglyceryl-20 docosabehenate/isostearate, polyglyceryl-20 docosabehenate/laurate, polyglyceryl-20 docosabehenate/oleate, polyglyceryl-20 heptadecabehenate/laurate, or a mixture of two or more thereof.

In another preferred embodiment, the amine (E) according to presently claimed invention is selected from primary amine, or a secondary amine.

In another preferred embodiment, the primary amine or secondary amine according to presently claimed invention is selected from methyl amine, ethyl amine, propyl amine, isopropyl amine, butyl amine, iso butyl amine, pentyl amine, isopentyl amine, neo pentyl amine, isoamyl amine, benzyl amine, 2-phenyl ethylamine, 1-phenyl ethyl amine, octyl amine, hexyl amine, heptyl amine, nonyl amine, decyl amine, ethylenediamine, 1,2-propylenediamine, ethanolamine, octylamine, 2-ethylhexylamine, 3-methoxypropylamine, 3-ethoxypropylamine, 3-hydroxypropylamine, decylamine, dodecylamine, tridecylamine, ditridecylamine, neopentanediamine, dihexylamine, di-(2-ethylhexyl)amine, 3,3′-dimethyl-4,4′-diaminodicyclohexylmethane, 4,4′-diaminodicyclohexylmethane, polyetheramine D 2000, polyetheramine D 230, polyetheramine D 400, polyetheramine T 403, polyetheramine T 5000, isophorone diamine, benzylamine, 2-(diethylamino)ethylamine, 2-(diisopropylamino)ethylamine, 3-(2-ethylhzexoxy)propylamine, 4,7,10-trioxytridecane-1,13-diamine, 4,9-dioxadodecane-1,12-diamine, or 1,12-diaminodecane, and most preferably the primary amine or secondary amine according to presently claimed invention is selected from ethylenediamine, 1,2-propylenediamine, ethanolamine, octylamine, 2-ethylhexylamine, 3-methoxypropylamine, 3-ethoxypropylamine, 3-hydroxypropylamine, decylamine, dodecylamine, tridecylamine, ditridecylamine, neopentanediamine, dihexylamine, di-(2-ethylhexyl)amine, 3,3′-dimethyl-4,4′-diaminodicyclohexylmethane, 4,4′-diaminodicyclohexylmethane, polyetheramine D 2000, polyetheramine D 230, polyetheramine D 400, polyetheramine T 403, polyetheramine T 5000, isophorone diamine, benzylamine, 2-(diethylamino)ethylamine, 2-(diisopropylamino)ethylamine, 3-(2-ethylhzexoxy)propylamine, 4,7,10-trioxytridecane-1,13-diamine, 4,9-dioxadodecane-1,12-diamine, or 1,12-diaminodecane.

In another preferred embodiment, the organic acid (F) according to presently claimed invention is selected from carboxylic acid, or sulfonic acid, or mixture thereof, more preferably the organic acid if a sulfonic acid.

In another preferred embodiment, the organic acid (F) according to presently claimed invention is an α-sulfo fatty ester or α-sulfo fatty acid.

In another preferred embodiment, the carboxylic acid according to presently claimed invention is selected from formic acid, acetic acid, trifluoroacetic acid, trichloro acetic acid, propionic acid, isopropionic acid, butyric acid, valeric acid, caproic acid, enanthic acid, caprylic acid, pelargonic acid, capric acid, cyclohexane dicarboxylic acid, oxalic acid, maleic acid, succinic acid, glutaric acid, adipic acid, pimelic acid, malic acid, pyruvic acid, citric acid, lauric acid, myristic acid, palmitic acid, stearic acid, oleic acid, benzoic acid, salicylic acid, gallic acid, toluic acid, phthalic acid, isophthalic acid, terephthalic acid, methoxy benzoic acid, decanoic acid, neodecanoic acid, lactic acid or a combination of two or more thereof.

In another preferred embodiment, the sulfonic acid according to presently claimed invention is selected from methane sulfonic acid, trifluoro methane sulfonic acid, trichloro methane sulfonic acid, toluene sulfonic acid, benzene sulfonic acid, naphthalene sulfonic acid, perfluoro butane sulfonic acid, perfluoro hexane sulfonic acid, dodecyl benzene sulfonic acid, perfluoro octane sulfonic acid, camphor sulfonic acid, vinyl sulfonic acid, or a combination of two or more thereof.

In another preferred embodiment, the Zr compound according to presently claimed invention is selected from ZrO₂, Zirconium halide, Zr(O(O)CR₆₁)₂, Zr(═O)(O(O)CR₆₁)₂, Zr(O(O)CR₆₁)₄, Zr(OCR₆₁)₄, Zr(O(O)CR₆₂C(O)O), or Zr(O(O)CR₆₂C(O)O)₂, Zr(O(O)₂SR₆₁)₂, Zr(O(O)₂SR₆₁)₄, Zr(O(O)₂SR₆₂S(O)₂O), or Zr(O(O)₂SR₆₂S(O)₂O)₂, wherein R₆₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl; and R₆₂ is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene.

In another preferred embodiment, the Zn compound according to presently claimed invention is selected from Zinc oxide, Zinc halide, Zn(O(O)CR₆₁)₂, or Zn(O(O)CR₆₂C(O)O), wherein R₆₁ and R₆₂ are as defined as above.

In another preferred embodiment, the Bi compound according to presently claimed invention is selected from Bismuth halide, Bismuth oxide, Bi(R₆₃)₂(O(O)CR₆₁), Bi(R₆₃)(O(O)CR₆₂C(O)O), or Bi(O(O)CR₆₁)₃, Bi(R₆₃)₂(O(O)₂SR₆₁), Bi(OR₆₃)₃, Bi(R₆₃)((O)₂SR₆₂S(O)₂O), or Bi(O(O)₂SR₆₁)₃, wherein R₆₁ and R₆₂ are as defined above, and R₆₃ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl.

In another preferred embodiment, the Zr(O(O)CR₆₁)₂, Zr(O)(O(O)CR₆₁)₂, Zr(O(O)CR₆₁)₄, Zr(OCR₆₁)₄, Zr(O(O)CR₆₂C(O)O), Zr(O(O)CR₆₂C(O)O)₂ Zr(O(O)₂SR₆₁)₂, Zr(O(O)₂SR₆₁)₄, Zr(O(O)₂SR₆₂S(O)₂O), or Zr(O(O)₂SR₆₂S(O)₂O)₂, compounds according to presently claimed invention are selected from zirconium acetate, zirconium octoate, zirconium 2-ethylhexanoate, zirconium decanoate, zirconium neodecanoate, bis(acetato-o)oxozirconium, bis(cyclopentadienyl)zirconium bis(trifluoromethanesulfonate) tetrahydrofuran adduct, zirconium(iv) acetylacetonate, zirconium(iv) tetrapropoxide, zirconium(iv) tetrabutoxide, zirconium 3-methyl-3-pentoxide, tetrakis(2-methyl-3-butene-2-oxy)zirconium, or tetrakis(1-methoxy-2-methyl-2-propoxy) zirconium

In another preferred embodiment, the Zn(O(O)CR₆₁)₂, or Zn(O(O)CR₆₂C(O)O) compounds according to presently claimed invention are selected from zinc neodecanoate, zinc octoate, zinc acetylacetonate, zinc oxalate, zinc acetate, zinc propionate, zinc valerate, zinc pivalate, zinc caprylate, zinc succinate, zinc bis(2-ethylhexanoate), zinc laurate, zinc myristate, zinc bis(trifluoroacetate), zinc stearate, zinc citrate, zinc gluconate, or benzeneacetic acid, α-octyl-α-phenyl-, zinc salt (2:1).

In another preferred embodiment, the Bi(R₆₃)₂(O(O)CR₆₁), Bi(R₆₃)(O(O)CR₆₂C(O)O), Bi(O(O)CR₆₁)₃, Bi(OR₆₃)₃, Bi(R₆₃)₂(O(O)₂SR₆₁), Bi(R₆₃)((O)₂SR₆₂S(O)₂O), or Bi(O(O)₂SR₆₁)₃ compounds according to presently claimed invention are selected from bismuth formate, bismuth sesquicarbonate, bismuth carbonate, bismuth octoate, bismuth octanoate, bismuth neodecanoate, bismuth(III) subsalicylate, bismuth neododecanoate, bismuth neooctanoate, bismuth, bismuth trineodecanoate, bismuth triacetate, bismuth tris(2-ethylhexanoate, bismuth triflate, bismuth beta naphthol, benzoic acid, 2,6-bis(diphenylmethyl)-4-methyl-, bismuth(3+) salt, benzeneacetic acid, α-octyl-α-phenyl-, bismuth(3+) salt, or bismuth carboxylate complex with CAS 2639142-12-2.

Within the context of the presently claimed invention, the term “alkyl”, as used herein, refers to an acyclic saturated aliphatic group, including linear or branched alkyl saturated hydrocarbon radicals, denoted by a general formula CₙH₂ₙ₊₁ and wherein n is the number of carbon atoms such as 1, 2, 3, 4, etc.

In a preferred embodiment, the unsubstituted linear C₁-C₂₄ alkyl is preferably selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl and tetracosyl; more preferably selected from the group consisting of hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl and tetracosyl; even more preferably selected from the group consisting of methyl, ethyl, propyl, butyl, pentyl, hexyl, heptyl, octyl, nonyl, decyl, undecyl, dodecyl, tridecyl, tetradecyl and pentadecyl; most preferably selected from the group consisting of methyl, ethyl, propyl, butyl, pentyl, hexyl, heptyl, octyl, nonyl, decyl, undecyl and dodecyl; and in particular selected from the group consisting of methyl, ethyl, propyl, butyl, pentyl, hexyl, heptyl, octyl, nonyl and decyl.

In a preferred embodiment, the unsubstituted branched C₁-C₂₄ alkyl is preferably selected from the group consisting of isopropyl, iso-butyl, neo-pentyl, 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, isodecyl, iso-dodecyl, iso-tetradecyl, iso-hexadecyl, iso-octadecyl and iso-eicosyl, more preferably selected from the group consisting of 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl, iso-eicosyl, 2-methyltricosyl, 2-ethyldocosyl, 3-ethylhenicosyl, 3-ethylicosyl, 4-propylhenicosyl, propylnonadecyl, 6-butyldodecyl and 5-ethylundecyl.

In a preferred embodiment, the substituted, linear or branched C₁-C₂₄ alkyl refers to a branched or linear saturated hydrocarbon group having C₁-C₂₄ carbon atoms substituted with functional groups selected from the group consisting of hydroxy, alkoxy, C(═O)—R, CN and SR, wherein R is selected from the group consisting of hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl and substituted or unsubstituted C₇-C₂₄ arylalkyl.

In a preferred embodiment, the substituted, linear or branched C₁-C₂₄ alkyl refers to a branched or linear saturated hydrocarbon group having C₁-C₂₄ carbon atoms substituted with functional groups selected from the group consisting of hydroxy, alkoxy, C(═O)—R, CN and SR, preferably selected from the group consisting of 1-hydroxy methyl, 1-methoxy methyl, 1-hydroxy ethyl, 1-hydroxy propyl, 1-hydroxy butyl, 1-hydroxy pentyl, 1-hydroxy hexyl, 1-hydroxy heptyl, 1-hydroxy octyl, 1-hydroxy nonyl, decyl, 1-hydroxy undecyl, 1-hydroxy dodecyl, 1-hydroxy tridecyl, 1-hydroxy tetradecyl, 1-hydroxy pentadecyl, 1-hydroxy hexadecyl, 1-hydroxy heptadecyl, 1-hydroxy octadecyl, 1-hydroxy nonadecyl, 1-hydroxy eicosyl, 1-hydroxy henicosyl, 1-hydroxy docosyl, 1-hydroxy tricosyl, 1-hydroxy tetracosyl, 1-methoxy methyl, 1-methoxy ethyl, 1-methoxy propyl, 1-methoxy butyl, 1-methoxy pentyl, 1-methoxy hexyl, 1-methoxy heptyl, 1-methoxy octyl, 1-methoxy nonyl, decyl, 1-methoxy undecyl, 1-methoxy dodecyl, 1-methoxy tridecyl, 1-methoxy tetradecyl, 1-methoxy pentadecyl, 1-methoxy hexadecyl, 1-methoxy heptadecyl, 1-methoxy octadecyl, 1-methoxy nonadecyl, 1-methoxy eicosyl, 1-methoxy henicosyl, 1-methoxy docosyl, 1-methoxy tricosyl, 1-methoxy tetracosyl, 2-methoxy propyl, 2-methoxy butyl, 2-methoxy pentyl, 2-methoxy hexyl, 2-methoxy heptyl, 2-methoxy octyl, 2-methoxy nonyl, decyl, 2-methoxy undecyl, 2-methoxy dodecyl, 2-methoxy tridecyl, 2-methoxy tetradecyl, 2-methoxy pentadecyl, 2-methoxy hexadecyl, 2-methoxy heptadecyl, 2-methoxy octadecyl, 2-methoxy nonadecyl, 2-methoxy eicosyl, 2-methoxy henicosyl, 2-methoxy docosyl, 2-methoxy tricosyl, 2-methoxy tetracosyl, 1-acetoxy methyl, 1-acetoxy ethyl, 1-acetoxy propyl, 1-acetoxy butyl, 1-acetoxy pentyl, 1-acetoxy hexyl, 1-acetoxy heptyl, 1-acetoxy octyl, 1-acetoxy nonyl, decyl, 1-acetoxy undecyl, 1-acetoxy dodecyl, 1-acetoxy tridecyl, 1-acetoxy tetradecyl, 1-acetoxy pentadecyl, 1-acetoxy hexadecyl, 1-acetoxy heptadecyl, 1-acetoxy octadecyl, 1-acetoxy nonadecyl, 1-acetoxy eicosyl, 1-acetoxy henicosyl, 1-acetoxy docosyl, 1-acetoxy tricosyl, 1-acetoxy tetracosyl, 1-cyano methyl, 1-cyano ethyl, 1-cyano propyl, 1-cyano butyl, 1-cyano pentyl, 1-cyano hexyl, 1-cyano heptyl, 1-cyano octyl, 1-cyano nonyl, decyl, 1-cyano undecyl, 1-cyano dodecyl, 1-cyano tridecyl, 1-cyano tetradecyl, 1-cyano pentadecyl, 1-cyano hexadecyl, 1-cyano heptadecyl, 1-cyano octadecyl, 1-cyano nonadecyl, 1-cyano eicosyl, 1-cyano henicosyl, 1-cyano docosyl, 1-cyano tricosyl, 1-cyano tetracosyl, 2-cyano propyl, 2-cyano butyl, 2-cyano pentyl, 2-cyano hexyl, 2-cyano heptyl, 2-cyano octyl, 2-cyano nonyl, decyl, 2-cyano undecyl, 2-cyano dodecyl, 2-cyano tridecyl, 2-cyano tetradecyl, 2-cyano pentadecyl, 2-cyano hexadecyl, 2-cyano heptadecyl, 2-cyano octadecyl, 2-cyano nonadecyl, 2-cyano eicosyl, 2-cyano henicosyl, 2-cyano docosyl, 2-cyano tricosyl, 2-cyano tetracosyl, 1-thioyl methyl, 1-thioyl ethyl, 1-thioyl propyl, 1-thioyl butyl, 1-thioyl pentyl, 1-thioyl hexyl, 1-thioyl heptyl, 1-thioyl octyl, 1-thioyl nonyl, decyl, 1-thioyl undecyl, 1-thioyl dodecyl, 1-thioyl tridecyl, 1-thioyl tetradecyl, 1-thioyl pentadecyl, 1-thioyl hexadecyl, 1-thioyl heptadecyl, 1-thioyl octadecyl, 1-thioyl nonadecyl, 1-thioyl eicosyl, 1-thioyl henicosyl, 1-thioyl docosyl, 1-thioyl tricosyl and 1-thioyl tetracosyl.

In a preferred embodiment, the term alkenyl denotes unsubstituted, linear C₂-C₂₄ alkenyl which is preferably selected from the group consisting of 1-propenyl, 1-butenyl, 1-pentenyl, 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl, 2-tetradecenyl, 1-pentadecenyl, 2-pentadecenyl, 1-hexadecenyl, 2-hexadecenyl, 1-heptadecenyl, 2-heptadecenyl, 1-octadecenyl, 2-octadecenyl, 1-nonadecenyl, 2-nonadecenyl, 1-eicosenyl and 2-eicosenyl, more preferably selected from 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl, 2-tetradecenyl, 1-pentadecenyl, 2-pentadecenyl, 1-hexadecenyl, 2-hexadecenyl, 1-heptadecenyl, 2-heptadecenyl, 1-octadecenyl, 2-octadecenyl, 1-nonadecenyl, 2-nonadecenyl, 1-eicosenyland 2-eicosenyl, 20-henicosenyl, 2-docosenyl, 6-tricosenyl and 2-tetracosenyl.

In a preferred embodiment, the unsubstituted branched C₂-C₂₄alkenyl is selected from the group consisting of isopropenyl, iso-butenyl, neo-pentenyl, 2-ethyl-hexenyl, 2-propyl-heptenyl, 2-butyl-octenyl, 2-pentyl-nonenyl, 2-hexyl-decenyl, iso-hexenyl, iso-heptenyl, iso-octenyl, iso-nonenyl, iso-decenyl, iso-dodecenyl, iso-tetradecenyl, iso-hexadecenyl, iso-octadecenyl, iso-eicosenyl, 2-methyl tricosenyl, 2-ethyl docosenyl, 3-ethylhenicosenyl, 3-ethyl icosenyl, 4-propylhenicosenyl, 4-propylnonadecenyl, 6-butyldodecenyl, 5-ethylundedcenyl, 1,4-hexadienyl, 1,3-hexadienyl, 2,5-hexadienyl, 3,5-hexadienyl, 2,4-hexadienyl, 1,3,5-hexatrienyl, 1,3,6-heptatrienyl, 1,4,7-octatrienyl or 2-methyl-1,3,5hexatrienyl, 1,3,5,7-octatetraenyl, 1,3,5,8-nonatetraenyl, 1,4,7,10-undecatetraenyl, 2-ethyl-1,3,6,8-nonatetraenyl, 2-ethenyl-1,3,5,8-nonatetraenyl, 1,3,5,7,9-decapentaenyl, 1,4,6,8,10-undecapentaenyl and 1,4,6,9,11-dodecapentaenyl.

In a preferred embodiment, the substituted, linear or branched C₂-C₂₄ alkenyl refers to a branched or a linear unsaturated hydrocarbon group having C₂-C₂₄ carbon atoms substituted with functional groups selected from, hydroxy, alkoxy, C(═O)—R, CN and SR; wherein R is hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl.

In a preferred embodiment the substituted, linear or branched C₂-C₂₄ alkenyl refers to a branched or an linear unsaturated hydrocarbon group having C₂-C₂₄carbon atoms substituted with functional groups selected from hydroxy, alkoxy, C(═O)—R, CN and SR; preferably selected from the group consisting of 2-hydroxy propenyl, 3-hydroxy butenyl, 3-hydroxy pentenyl, 5-hydroxy hexenyl, 7-hydroxy heptenyl, 3-hydroxy octenyl, 5-hydroxy nonenyl, decyl, 11-hydroxy undecenyl, 9-hydroxy dodecenyl, 6-hydroxy tridecenyl, 4-hydroxy tetradecenyl, 6-hydroxy pentadecenyl, 3-hydroxy hexadecenyl, 2-hydroxy heptadecenyl, 7-hydroxy octadecenyl, 6-hydroxy nonadecenyl, 4-hydroxy eicosenyl, 2-hydroxy henicosenyl, 3-hydroxy docosenyl, 2-hydroxy tricosenyl, 23-hydroxy tetracosenyl, 1-methoxy ethenyl, 2-methoxy propenyl, 4-methoxy butenyl, 3-methoxy pentenyl, 5-methoxy hexenyl, 2-methoxy heptenyl, 5-methoxy octenyl, 3-methoxy nonenyl, 6-methoxy undecenyl, 1-methoxy dodec-2-enyl, 1-methoxy tridec-5-enyl, 3-methoxy tetradic-5-enyl, 3-methoxy pentade-12-encyl, 10-methoxy hexadec-15-enyl, 12-methoxy heptadic-16-enyl, 1-methoxy octadec-3-enyl, 1-methoxy nonadec-2-enyl, 1-methoxy eicos-20-enyl, 1-methoxy henicos-2-enyl, 1-methoxy docos-4-enyl, 1-methoxy tricos-22-enyl, 1-methoxy tetracos-23-enyl, 2-methoxy prop-1-enyl, 2-methoxy but-1-enyl, 2-methoxy pent-4-enyl, 2-methoxy hex-2-enyl, 2-methoxy hept-3-enyl, 2-methoxy oct-7-enyl, 2-methoxy non-5-enyl, 2-methoxy undec-10-enyl, 2-methoxy dodec-4-enyl, 2-methoxy tridec-12-enyl, 2-methoxy tetradic-10-enyl, 2-methoxy pentadec-14-enyl, 2-methoxy hexadec-1-enyl, 2-methoxy heptadic-1-enyl, 2-methoxy octadic-12-enyl, 2-methoxy nonadec-10-enyl, 2-methoxy eicos-18-enyl, 2-methoxy henicos-2-enyl, 2-methoxy docos-3-enyl, 20-methoxy tricos-2-enyl, 21-methoxy tetracos-4-enyl, 1-acetoxy ethenyl, 1-acetoxy prop-1-enyl, 1-acetoxy but-2-enyl, 1-acetoxy pent-4-enyl, 1-acetoxy hex-2-enyl, 1-acetoxy hept-1-enyl, 1-acetoxy oct-7-enyl, 1-acetoxy non-2-enyl, 5-acetoxy dec-3-enyl, 1-acetoxy undec-10-enyl, 1-acetoxy dodec-2-enyl, 1-acetoxy tridec-12-enyl, 10-acetoxy tetradec-2-enyl, 15-acetoxy pentadec-2-enyl, 10-acetoxy hexadec-2-enyl, 11-acetoxy heptadec-1-enyl, 13-acetoxy octadec-2-enyl, 1-acetoxy nonadec-14-enyl, 20-acetoxy eicos-19-enyl, 1-acetoxy henicos-2-enyl, 1-acetoxy docos-10-enyl, 1-acetoxy tricos-22-enyl, 1-acetoxy tetracos-23-enyl, 1-cyano eth-1-enyl, 1-cyano prop-2-enyl, 1-cyano but-2-enyl, 1-cyano pent-3-enyl, 1-cyano hex-5-enyl, 1-cyano hept-6-enyl, 1-cyano oct-2-enyl, 1-cyano non-3-enyl, 11-cyano undec-2-enyl, 10-cyano dodec-2-enyl, 10-cyano tridec-12-enyl, 1-cyano tetradec-3-enyl, 1-cyano pentadec-14-enyl, 1-cyano hexadec-15-enyl, 1-cyano heptadec-2-enyl, 1-cyano octadec-3-enyl, 1-cyano nonadec-18-enyl, 1-cyano eicos-10-enyl, 1-cyano henicos-20-enyl, 15-cyano docos-3-enyl, 1-cyano tricos-20-enyl, 1-cyano tetracos-2-enyl, 2-cyano prop-2-enyl, 2-cyano but-1-enyl, 2-cyano pent-1-enyl, 2-cyano hex-3-enyl, 2-cyano hept-6-enyl, 2-cyano oct-1-enyl, 2-cyano non-8-enyl, 2-cyano undec-10-enyl, 2-cyano dodec-1-enyl, 2-cyano tridec-12-enyl, 2-cyano tetradec-10-enyl, 2-cyano pentadec-3-enyl, 2-cyano hexadec-2-enyl, 2-cyano heptadec-1-enyl, 2-cyano octadec-12-enyl, 2-cyano nonadec-15-enyl, 2-cyano eicos-1-enyl, 2-cyano henicos-5-enyl, 2-cyano docos-20-enyl, 2-cyano tricos-22-enyl, 2-cyano tetracos-20-enyl, 1-thionyl eth-1-enyl, 1-thionyl prop-2-enyl, 1-thionyl but-2-enyl, 1-thionyl pent-4-enyl, 1-thionyl hex-2-enyl, 1-thionyl hept-5-enyl, 1-thionyl oct-3-enyl, 1-thionyl non-5-enyl, 1-thionyl undec-10-enyl, 1-thionyl dodec-11-enyl, 1-thionyl tridec-2-enyl, 1-thionyl tetradec-4-enyl, 1-thionyl pentadec-5-enyl, 1-thionyl hexadec-3-enyl, 1-thionyl heptadec-2-enyl, 1-thionyl octadec-3-enyl, 1-thionyl nonadec-15-enyl, 1-thionyl eicos-18-enyl, 1-thionyl henicos-20-enyl, 1-thionyl docos-21-enyl, 1-thionyl tricos-20-enyl and 1-thionyl tetracos-22-enyl.

In a preferred embodiment, the substituted or unsubstituted C₅-C₂₄ cycloalkyl refers to a monocyclic and bicyclic 5- to 24-membered saturated cycloaliphatic radical. Representative examples of unsubstituted or branched C₅-C₂₄ monocyclic and bicyclic cycloalkyl include, but are not limited to, cyclopentyl, cyclohexyl, cycloheptyl, cyclooctyl, bicyclo[2.2.1]heptyl, and bicyclo[3.1.1]heptyl.

In another preferred embodiment, the C₅-C₂₄ monocyclic and bicyclic cycloalkyl can be further branched with one or more equal or different alkyl groups such as methyl, ethyl, n-propyl, iso-propyl, n-butyl, iso-butyl, n-pentyl, iso-pentyl, neo-pentyl etc. The representative examples of branched C₃-C₁₀ monocyclic and bicyclic cycloalkyl include, but are not limited to, methyl cyclohexyl and dimethyl cyclohexyl.

In a preferred embodiment, the unsubstituted or substituted C₅-C₂₄ cycloalkenyl refers to a monocyclic and bicyclic 5- to 24-membered unsaturated cycloaliphatic radical which comprises one or more double bonds. Representative examples of C₅-C₂₄ cycloalkenyl include, but are not limited to, cyclopentenyl, cyclohexenyl, cycloheptenyl, cyclooctenyl, cyclononenyl or cyclodecenyl. These radicals can be branched with one or more equal or different alkyl radical, preferably with methyl, ethyl, n-propyl or iso-propyl. The representative examples of branched C₅-C₂₄ monocyclic and bicyclic cycloalkenyl include, but are not limited to, methyl cyclohexenyl and dimethyl cyclohexenyl.

In a preferred embodiment, the substituted or unsubstituted C₆-C₂₄ aryl may have more than one aromatic ring. The representative examples for substituted and unsubstituted C₆-C₂₄ aryl include phenyl, naphthyl, anthracenyl, tetraphenyl, phenalenyl and phenanthrenyl.

In a preferred embodiment, the arylalkyl refers to an aryl ring attached to an alkyl chain. The representative examples for the arylalkyl include, but are not limited to, 1-phenylmethyl, 1-phenylethyl, 1-phenylpropyl, 1-phenylbutyl, 1-methyl-1-phenyl-propyl, 3-phenylpropyl, 4-phenylbutyl, 3-phenylbutyl and 2-methyl-3-phenyl-propyl.

In a preferred embodiment, the substituted C₆-C₂₄ aryl refers to an aromatic ring having substitution at different positions. The C₆-C₂₄ aryl may have more than one aromatic ring. The representative examples for substituted and unsubstituted C₆-C₂₄ aryl include tolyl, xylyl, 2-hydroxy-phenyl, 2,3-dihydroxyphenyl, 2-methoxy phenyl, 2-hydroxy-4-methoxyphenyl, 2,4-dimethoxyphenyl, 2-chlorophenyl, 2-chloro-4-hydroxyphenyl, 2-chloro-4-methoxy phenyl, 3-chloro-4-methoxyphenyl, 2-methyl-4-methoxy-6-chlorophenyl and 2-acetyl-4-hydroxy-phenyl.

Within the context of the presently claimed invention, the term alkylene refers to an acyclic saturated hydrocarbon chains, which combine different moieties.

The alkylene refers to linear unsubstituted C₁ to C₃₀ carbon atoms, selected from, but not limited to, —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—CH₂—CH₂—CH₂—, —CH₂—CH₂—CH₂—CH₂—CH₂—CH₂—CH₂—, —CH₂—CH₂—CH₂—CH₂—CH₂—CH₂—CH₂—CH₂—, —CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, or —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—.

In another preferred embodiment the branched unsubstituted alkylene selected from, but not limited to, —CH₂—C(CH₃)H—, —CH₂—C(CH₃)H—CH₂—, —CH₂—CH₂—C(CH₃)H—CH₂—, —C(CH₃)₂—CH₂—CH₂—C(CH₃)H—CH₂—, —CH₂—C(CH₃)H—CH₂—CH₂—CH₂—CH₂—, —CH₂—C(CH₃)H—CH₂—CH₂—CH₂—CH₂—CH₂—, —CH₂—CH₂—CH₂—CH₂—CH₂—CH₂—CH₂—C(CH₃)H—, —C(CH₃)H—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—, —C(CH₃)₂—CH₂—CH₂—CH₂—CH₂—CH₂—CH₂—CH₂—CH₂—CH₂—CH₂—, —CH₂—C(CH₃)₂—CH₂—CH₂—CH₂—CH₂—CH₂—CH₂—CH₂—CH₂—CH₂—CH₂—, —CH₂—C(CH₃)H—CH₂—CH₂—CH₂—CH₂—CH₂—CH₂—CH₂—CH₂—CH₂—CH₂—CH₂—, —C(CH₃)₂—CH₂—CH₂—CH₂—CH₂—CH₂—CH₂—CH₂—CH₂—CH₂—CH₂—CH₂—CH₂—CH₂—, —CH₂—CH₂—CH₂—C(CH₃)H—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—, —CH₂—C(CH₃)H—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—C(CH₃)H—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CHO—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, or —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—.

In another preferred embodiment, the mole ratio of compound of formula (I) to the total amount of alcohol (D) to amine (E) is in the range of 50:0.1 to 0.1:50, more preferably the mole ratio of compound of formula (I) to the total amount of alcohol (D) or amine (E) is in the range of 30:0.1 to 0.1:30, even more preferably the mole ratio of compound of formula (I) to the total amount of alcohol (D) or amine (E) is in the range of 20:0.1 to 0.1:20, most preferably the mole ratio of compound of formula (I) to the total amount of alcohol (D) or amine (E) is in the range of 10:0.1 to 0.1:10, and in particular preferably the mole ratio of compound of formula (I) to the total amount of alcohol (D) or amine (E) is in the range of 5.0:0.1 to 0.1:5.0.

In another preferred embodiment, wherein the catalyst in the reaction is present in an amount in the range of 0.0001 to 30 wt. % based on total weight of compounds of formula (I), more preferably the catalyst in the reaction is present in a total amount in the range of 0.01 to 10 wt. % based on total weight of compounds of formula (I), even more preferably the catalyst in the reaction is present in a total amount in the range of 0.01 to 5 wt. % based on total weight of compounds of formula (I), most preferably the catalyst in the reaction is present in a total amount in the range of 0.01 to 3.0 wt. % based on total weight of compounds of formula (I), and in particular the catalyst in the reaction is present in a total amount in the range of 0.01 to 1 wt. % based on total weight of compounds of formula (I).

In another preferred embodiment, wherein the catalyst in the reaction is present in an amount in the range of 0.0001 to 2.0 mole equivalent based on compounds of formula (I), more preferably the catalyst in the reaction is present in a total amount in the range of 0.0001 to 1.0 mole equivalent based on compounds of formula (I), even more preferably the catalyst in the reaction is present in a total amount in the range of 0.001 to 1.0 mole equivalent based on compounds of formula (I), most preferably the catalyst in the reaction is present in a total amount in the range of 0.001 to 0.5 mole equivalent based on compounds of formula (I), and in particular the catalyst in the reaction is present in a total amount in the range of 0.001 to 0.1 mole equivalent based on compounds of formula (I).

In another preferred embodiment, the organic acid (F) in the step c) is present in an amount of 0.001 to 30 wt. % based on total weight of compound of formula (I), more preferably the organic acid (F) in step c) is present in an amount of 0.001 to 10.0 wt. % based on total weight of compound of formula (I), even more preferably the organic acid (F) in step c) is present in an amount of 0.001 to 5.0 wt. % based on total weight of compound of formula (I), most preferably the organic acid (F) in step c) is present in an amount of 0.001 to 1.0 wt. % based on total weight of compound of formula (I), and in particular the organic acid (F) in step c) is present in an amount of 0.001 to 0.5 wt. % based on total weight of compound of formula (I).

In another preferred embodiment, the organic acid (F) in the step c) is present in an amount of 0.0001 to 2.0 mole equivalent based on compounds of formula (I), more preferably the organic acid (F) in step c) is present in an amount of 0.0001 to 1.0 mole equivalent based on compounds of formula (I), even more preferably the organic acid (F) in step c) is present in an amount of 0.001 to 0.5 mole equivalent based on compounds of formula (I), most preferably the organic acid (F) in step c) is present in an amount of 0.001 to 0.2 mole equivalent based on compounds of formula (I), and in particular the organic acid (F) in step c) is present in an amount of 0.001 to 0.1 mole equivalent based on compounds of formula (I).

In another preferred embodiment, the step d) is carried out in presence of a solvent or in the absence of a solvent.

In another preferred embodiment, the solvent is selected from ethers, lactones, carbonates, sulfones, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof, more preferably the solvent is selected from ethers, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof, even more preferably the solvent is selected from ethers, N, N-dimethylformamide, N-methyl-pyrrolidone, N-ethyl-pyrrolidone, aromatic hydrocarbons, dichloroethane, halogenated aromatic hydrocarbon, or a combination of two or more thereof, most preferably the solvent is selected from ethers, N, N-dimethylformamide, N-methyl-pyrrolidone, N-ethyl-pyrrolidone, aromatic hydrocarbons, halogenated aromatic hydrocarbon, or a combination of two or more thereof, and in particular the solvent is selected from ethers, aromatic hydrocarbons, halogenated aromatic hydrocarbon, or a combination thereof.

In another preferred embodiment, the step d) is carried out in presence of a solvent in an amount in the range of 0.5 to 20 times based of total amount of formula (I), more preferably the step d) is carried out in presence of a solvent in an amount in the range of 0.5 to 10 times based of total amount of formula (I), even more preferably the step d) is carried out in presence of a solvent in an amount in the range of 0.5 to 5.0 times based of total amount of formula (I), most preferably the step d) is carried out in presence of a solvent in an amount in the range of 0.5 to 3.0 times based of total amount of formula (I), and in particular the step d) is carried out in presence of a solvent in an amount in the range of 0.5 to 2.0 times based of total amount of formula (I).

In another preferred embodiment, the step d) is carried out at a temperature in the range of ≥30° C. to ≤250° C., more preferably the step d) is carried out at a temperature in the range of ≥100° C. to ≤220° C., even more preferably the step d) is carried out at a temperature in the range of ≥130° C. to ≤220° C. most preferably the step d) is carried out at a temperature in the range of ≥150° C. to ≤220° C. and in particular the step d) is carried out at a temperature in the range of ≥150° C. to ≤200° C.

In another preferred embodiment, the pH of the step c) and/or step d) is maintained 8.0, more preferably the pH of the step c) and/or step d) is maintained in the range of ≥0.0 to 8.0, even more preferably the pH of the step c) and/or step d) is maintained in the range of ≥0.0 to 4.0, most preferably the pH of the step c) and/or step d) is maintained in the range of ≥0.5 to 3.5 and in particular preferably the pH of the step c) and/or step d) is maintained in the range of ≥0.5 to 3.0.

In another preferred embodiment, the process according to the presently claimed invention further comprises a step e) of isolation of an ester or an amide of a compound of formula (I) from the reaction mixture. The ester or the amide of the compound of formula (I) formed in the reaction is isolated by any method known in the art selected from the group consisting of chemical separation, acid-base neutralization, distillation, evaporation, column chromatography, filtration, concentration, crystallization and re-crystallization or a combination thereof. A person skilled in the art is aware of such techniques.

In another embodiment the presently claimed invention is directed to an ester or an amide of a compound of formula (I) obtained according to the presently claimed process, preferably the ester or the amide of the compound of formula (I) obtained according to the presently claimed process comprises Zr, Zn, Bi, or combination of two or more in free form or a compound form in an amount in the range of 0.0001 to 5.0 wt. % based on total amount of the ester or the amide of the compound of formula (I), more preferably the ester or the amide of the compound of formula (I) obtained according to the presently claimed process comprises Zr, Zn, Bi, or combination of two or more in free form or a compound form in an amount in the range of 0.0001 to 1.0 wt. % based on total amount of the ester or the amide of the compound of formula (I), even more preferably the ester or the amide of the compound of formula (I) obtained according to the presently claimed process comprises Zr, Zn, Bi, or combination of two or more in free form or a compound form in an amount in the range of 0.0001 to 0.5 wt. % based on total amount of the ester or the amide of the compound of formula (I), most preferably the ester or the amide of the compound of formula (I) obtained according to the presently claimed process comprises Zr, Zn, Bi, or combination of two or more in free form or a compound form in an amount in the range of 0.0001 to 0.1 wt. % based on total amount of the ester or the amide of the compound of formula (I), and in particular the ester or the amide of the compound of compound of formula (I) obtained according to the presently claimed process comprises Zr, Zn, Bi, or combination of two or more in free form or a compound form in an amount in the range of 0.0001 to 0.01 wt. % based on total amount of the ester or the amide of the compound of formula (I).

In another preferred embodiment, the ester or the amide of the compound of formula (I) obtained according to the presently claimed process comprises Zr, Zn, Bi, or combination of two or more in free form or a compound form in an amount in the range of 10 ppm to 5000 ppm based on total amount of the ester or the amide of the compound of formula (I), more preferably the ester or the amide of the compound of formula (I) obtained according to the presently claimed process comprises Zr, Zn, Bi, or combination of two or more in free form or a compound form in an amount in the range of 10 ppm to 3000 ppm based on total amount of the ester or the amide of the compound of formula (I), even more preferably the ester or the amide of the compound of formula (I) obtained according to the presently claimed process comprises Zr, Zn, Bi, or combination of two or more in free form or a compound form in an amount in the range of 100 ppm to 2000 ppm based on total amount of the ester or the amide of the compound of formula (I), most preferably the ester or the amide of the compound of formula (I) obtained according to the presently claimed process comprises Zr, Zn, Bi, or combination of two or more in free form or a compound form in an amount in the range of 200 ppm to 2000 ppm based on total amount of the ester or the amide of the compound of formula (I), and in particular the ester or the amide of the compound of formula (I) obtained according to the presently claimed process comprises Zr, Zn, Bi, or combination of two or more in free form or a compound form in an amount in the range of 500 ppm to 150° ppm based on total amount of the compound of formula (I).

In another embodiment the presently claimed invention is directed to an ester or an amide of a compound of formula (I) comprising Zr, Zn, Bi, or combination of two or more in free form or a compound form in an amount in the range of 0.0001 to 5.0 wt. % based on total amount of the ester or the amide of the compound of formula (I), more preferably the ester or the amide of the compound of formula (I) comprising Zr, Zn, Bi, or combination of two or more in free form or a compound form in an amount in the range of 0.0001 to 1.0 wt. % based on total amount of the ester or the amide of the compound of formula (I), even more preferably the ester or the amide of the compound of formula (I) comprising Zr, Zn, Bi, or combination of two or more in free form or a compound form in an amount in the range of 0.0001 to 0.5 wt. % based on total amount of the ester or the amide of the compound of formula (I), most preferably the ester or the amide of the compound of formula (I) comprising Zr, Zn, Bi, or combination of two or more in free form or a compound form in an amount in the range of 0.0001 to 0.1 wt. % based on total amount of the ester or the amide of the compound of formula (I), and in particular the ester or the amide of the compound of compound of formula (I) comprising Zr, Zn, Bi, or combination of two or more in free form or a compound form in an amount in the range of 0.0001 to 0.01 wt. % based on total amount of the ester or the amide of the compound of formula (I).

In another preferred embodiment, the ester or the amide of the compound of formula (I) comprising Zr, Zn, Bi, or combination of two or more in free form or a compound form in an amount in the range of 10 ppm to 5000 ppm based on total amount of the ester or the amide of the compound of formula (I), more preferably the ester or the amide of the compound of formula (I) comprising Zr, Zn, Bi, or combination of two or more in free form or a compound form in an amount in the range of 10 ppm to 3000 ppm based on total amount of the ester or the amide of the compound of formula (I), even more preferably the ester or the amide of the compound of formula (I) comprising Zr, Zn, Bi, or combination of two or more in free form or a compound form in an amount in the range of 100 ppm to 2000 ppm based on total amount of the ester or the amide of the compound of formula (I), most preferably the ester or the amide of the compound of formula (I) comprising Zr, Zn, Bi, or combination of two or more in free form or a compound form in an amount in the range of 200 ppm to 2000 ppm based on total amount of the ester or the amide of the compound of formula (I), and in particular the ester or the amide of the compound of formula (I) comprising Zr, Zn, Bi, or combination of two or more in free form or a compound form in an amount in the range of 500 ppm to 150° ppm based on total amount of the compound of formula (I).

In another embodiment the presently claimed invention is directed to a composition comprising an ester or an amide of a compound of formula (I), preferably the presently claimed invention is directed to a composition comprising an ester or an amide of a compound of formula (I) obtained according to the presently claimed process.

In another embodiment the presently claimed invention is directed to the use of an ester or an amide of a compound of formula (I), preferably the presently claimed invention is directed to a composition comprising an ester or an amide of a compound of formula (I) obtained according to the presently claimed process.

In another embodiment the presently claimed invention is directed to a method of stabilizing coating/composition against UV light comprising at least the steps of:
a) proving an ester or an amide of a compound of formula (I) or a composition comprising an ester or an amide of a compound of formula (I).

The presently claimed invention is associated with at least one of the following advantages:
The process is efficiently reducing the time of the reaction with environmentally benign catalyst.
The reaction time can be further reduced by addition of organic acid, thus essentially improves the space time yield and saves energy.
The process led to reduction in use of in environmental hazardous metal. The UV absorber compound according to presently claimed invention display similar UV absorbance as prior art product even though it comprises residual catalyst metals.

### Embodiments

In the following, there is provided a list of embodiments to further illustrate the present disclosure without intending to limit the disclosure to the specific embodiments listed below.
1. A process for obtaining an ester or an amide of a compound of formula (I) comprising at least the step of:
   a) providing at least one compound of formula (I),

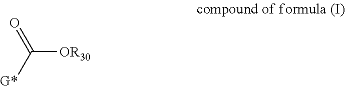

wherein
G* is selected from formula (A), or formula (B)

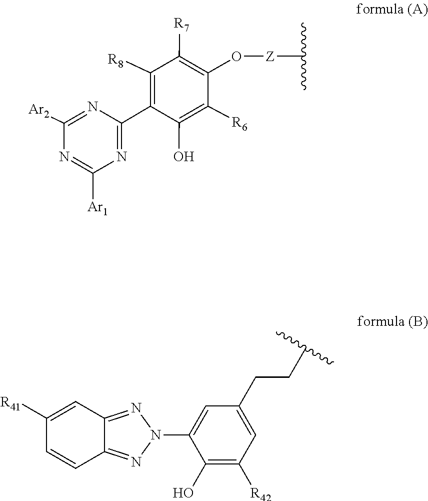

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C), or a moiety of formula (M),

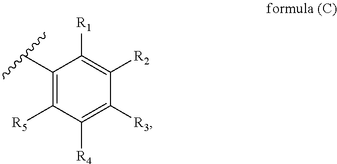

wherein R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, OR,
R selected from hydrogen, substituted or unsubstituted, linear or branched C₁-C₂₄ alkyl,

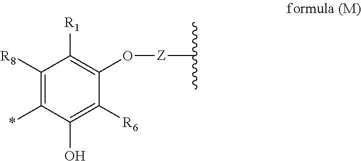

wherein the dotted line is a single bond between formula (M) and the triazinyl ring of formula (A), Z is as defined as above;
R₆, R₇ and R₈ are independently of each other selected from hydrogen, substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
b) providing at least one alcohol (D) or an amine (E) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.
2. The process according to embodiment 1,
   wherein R₃₀ is selected from hydrogen, substituted or unsubstituted, linear or branched C₁-C₂₄ alkyl, substituted or unsubstituted, linear or branched C₂-C₂₄ alkenyl, or substituted or unsubstituted C₅-C₂₄ cycloalkyl,
      G* is

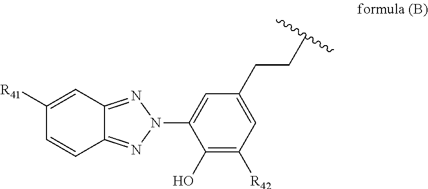

wherein R₄₁, and R₄₂ are independently of each other selected from hydrogen, halogen, substituted or unsubstituted, linear or branched C₁-C₂₄ alkyl, substituted or unsubstituted, linear or branched C₂-C₂₄alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.
3. The process according to any one of the embodiments 1 to 2, wherein the Zr compounds are selected from ZrO₂, Zirconium halide, Zr(O(O)CR₆₁)₂, Zr(═O)(O(O)CR₆₁)₂, Zr(O(O)CR₆₁)₄, Zr(O(O)CR₆₂C(O)O), or Zr(O(O)CR₆₂C(O)O)₂, Zr(O(O)₂SR₆₁)₂, Zr(O(O)₂SR₆₁)₄, Zr(O(O)₂SR₆₂S(O)₂O), or Zr(O(O)₂SR₆₂S(O)₂O)₂, wherein R₆₁ is selected form substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl; and R₆₂ is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene.
4. The process according to any one of the embodiments 1 to 2, wherein the Zn compound is selected from Zinc oxide, Zinc halide, Zn(O(O)CR₆₁)₂, or Zn(O(O)CR₆₂C(O)O), wherein R₆₁ and R₆₂ are as defined in embodiment 3.
5. The process according to any one of the embodiments 1 to 2, wherein the Bi compound is selected from Bismuth halide, Bismuth oxide, Bi(R₆₃)₂(O(O)CR₆₁), Bi(R₆₃)(O(O)CR₆₂C(O)O), or Bi(O(O)CR₆₁)₃, Bi(OR₆₃)₃, Bi(R₆₃)₂(O(O)₂SR₆₁), Bi(R₆₃)((O)₂SR₆₂S(O)₂O), or Bi(O(O)₂SR₆₁)₃, wherein R₆₁ and R₆₂ are as defined in embodiment 5, and R₆₃ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl.
6. The process according to any one of the embodiments 1 to 5, wherein the at least one organic acid (F) is different from the acid of compound of formula (I).
7. The process according to any one of the embodiments 1 to 6, wherein organic acid (F) is selected from carboxylic acid, or sulfonic acid.
8. The process according to embodiment 7, wherein the organic acid (F) is sulfonic acid.
9. The process according to embodiment 7, wherein the carboxylic acid is selected from formic acid, acetic acid, trifluoroacetic acid, trichloro acetic acid, propionic acid, isopropionic acid, butyric acid, valeric acid, caproic acid, enanthic acid, caprylic acid, pelargonic acid, capric acid, cyclohexane dicarboxylic acid, oxalic acid, maleic acid, succinic acid, glutaric acid, adipic acid, pimelic acid, malic acid, pyruvic acid, citric acid, lauric acid, myristic acid, palmitic acid, stearic acid, oleic acid, benzoic acid, salicylic acid, gallic acid, toluic acid, phthalic acid, isophthalic acid, terephthalic acid, methoxy benzoic acid, decanoic acid, neodecanoic acid, lactic acid or a combination of two or more thereof.
10. The process according to any one of the embodiments 7 to 8, wherein the sulfonic acid is selected from methane sulfonic acid, trifluoro methane sulfonic acid, trichloro methane sulfonic caid, toluene sulfonic acid, benzene sulfonic acid, naphthalene sulfonic acid, perfluoro butane sulfonic acid, perfluoro hexane sulfonic acid, dodecyl benzene sulfonic acid, perfluoro octane sulfonic acid, camphor sulfonic acid, vinyl sulfonic acid, or a combination of two or more thereof.
11. The process according to any one of the embodiments 1 to 10, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), 6-methylheptyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxoethoxy)phenyl]-6-(4-methoxyphenyl)-1,3,5-triazin-2-yl]phenoxy]propanoate, or a combination of two or more thereof.
12. The process according to embodiment 11, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.
13. The process according to any one of the embodiments 1 to 12, wherein the alcohol (D) is a monohydric or a polyhydric alcohol.
14. The process according to anyone of the embodiments 1 to 13, wherein the amine (E) is a compound having at least one primary or at least one secondary amine functional group.
15. The process according to anyone of the embodiments 1 to 14, wherein the alcohol (D) is selected from pentaerythritol, trimethylolpropane, ethylene glycol, substituted or unsubstituted C₁₋₃₀ alkanol having one or more hydroxy functional groups, substituted or unsubstituted C₃₋₃₀ alkenol having one or more hydroxy functional groups, substituted or unsubstituted C₃₋₃₀ heteroalkanol having one or more hydroxy functional groups, substituted or unsubstituted C₄₋₃₀ hetero alkenol having one or more hydroxy functional groups, substituted or unsubstituted aromatic phenol having one or more hydroxy functional groups, substituted or unsubstituted aralkyl having one or more hydroxy functional groups, substituted or unsubstituted cyclic alcohol having one or more hydroxy functional groups, polyalkyleneoxide having one or more hydroxy functional groups, substituted or unsubstituted polyglycerols, or a combination of two or more thereof.
16. The process according to anyone of the embodiments 1 to 17, wherein the alcohol (D) is selected from pentaerythritol, trimethylolpropane, ethylene glycol, polyethylene glycol, 2-ethyl hexanol, octanol, isooctanol, ditrimethylolethane, ditrimethylolpropane, tristrimethylolpropane, 1,2-propanediol, 1,3-propanediol, diethylene glycol, dipropylene glycol, triethylene glycol, tetraethylene glycol, 1,2-butanediol, 1,4-butanediol, 1,3-butylethylpropanediol, 1,3-methylpropanediol, 1,5-pentanediol, bis(1,4-hydroxymethyl)cyclohexane, glycerol, hexanediol, neopentyl glycol, trimethylolethane, trimethylolpropane, pentaerythritol, bisphenol A, bisphenol B, bisphenol C, bisphenol F, norbornylene glycol, 1,4-benzyldimethanol, 1,4-benzyldiethanol, 2,4-dimethyl-2-ethylhexane-1,3-diol, 1,4-butylene glycol, 2,3-butylene glycol, norbomylene glycol, 1,4-benzyldimethanol, 1,4-benzyldiethanol, 2,4-dimethyl-2-ethylhexane-1,3-diol, 1,4- and 2,3-butylene glycol, di-(3-hydroxyethylbutanediol, 1,5-pentanediol, 1,6-hexanediol, 1,8-octanediol, decanediol, dodecanediol, neopentyl glycol, cyclohexanediol, 3(4),8(9)-bis(hydroxymethyl)-tricyclo[5.2.1.02-6]decane (dicidol), 2,2-bis-(4-hydroxycyclohexyl)propane, 2,2-bis-[4-((3-hydroxyethoxy)phenyl]propane, 2-methylpropane-1,3-diol, 2-methylpentane-1,5-diol, 2,2,4(2,4,4)trimethylhexane-1,6-diol, hexane-1,2,6-triol, butane-1,2,4-triol, tris((3-hydroxyethyl) isocyanurate, mannitol, sorbitol, polypropylene glycols, polybutylene glycols, xylylene glycol, neopentyl glycol hydroxypivalate, hydroxyacrylates, polyglycerol, or a combination of two or more thereof.
17. The process according to anyone of the embodiments 1 to 16, wherein the amine (E) has at least one primary or secondary amine functional group. is selected from selected from methyl amine, ethyl amine, propyl amine, isopropyl amine, butyl amine, iso butyl amine, pentyl amine, isopentyl amine, neo pentyl amine, isoamyl amine, benzyl amine, 2-phenyl ethylamine, 1-phenyl ethyl amine, octyl amine, hexyl amine, heptyl amine, nonyl amine, decyl amine, ethylenediamine, 1,2-propylenediamine, ethanolamine, octylamine, 2-ethylhexylamine, 3-methoxypropylamine, 3-ethoxypropylamine, 3-hydroxypropylamine, decylamine, dodecylamine, tridecylamine, ditridecylamine, neopentanediamine, dihexylamine, di-(2-ethylhexyl)amine, 3,3′-dimethyl-4,4′-diaminodicyclohexylmethane, 4,4′-diaminodicyclohexylmethane, Polyetheramine D 2000, Polyetheramine D 230, Polyetheramine D 400, Polyetheramine T 403, Polyetheramine T 5000, isophorone diamine, benzylamine, 2-(diethylamino)ethylamine, 2-(diisopropylamino)ethylamine, 3-(2-ethylhzexoxy)propylamine, 4,7,10-trioxytridecane-1,13-diamine, 4,9-dioxadodecane-1,12-diamine, or 1,12-diaminodecane.
18. The process according to any one of the embodiments 1 to 17, wherein the mole ratio of compound of formula (I) to the total amount of alcohol (D) or amine (E) is in the range of 50:0.1 to 0.1:50.
19. The process according to embodiment 18, wherein the mole ratio of compound of formula (I) to the total amount of alcohol (D) or amine (E) is in the range of 20:0.1 to 0.1:20.
20. The process according to any one of the embodiments 1 to 19, wherein the catalyst in the reaction is present in an amount in the range of 0.0001 to 30 wt. % based on total weight of compounds of formula (O).
21. The process according to embodiment 20, wherein the catalyst in the reaction is present in a total amount in the range of 0.01 to 5 wt. % based on total weight of compounds of formula (I).
22. The process according to any one of the embodiments 1 to 21, wherein the organic acid (F) in the step c) is present in an amount of 0.001 to 30 wt. % based on total weight of compound of formula (I).
23. The process according to embodiment 22, wherein the organic acid (F) in step c) is present in an amount of 0.001 to 1.0 wt. % based on total weight of compound of formula (I).
24. The process according to any one of the embodiments 1 to 23, wherein the step d) is carried out in presence of a solvent or in the absence of a solvent.
25. The process according to embodiment 24, wherein the solvent is selected from ethers, lactones, carbonates, sulfones, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof.
26. The process according to any one of the embodiments 1 to 25, wherein the step d) is carried out in presence of a solvent in an amount in the range of 0.5 to 20 times based of total amount of formula (I).
27. The process according to any one of the embodiments 1 to 26, wherein the step d) is carried out at a temperature in the range of ≥30° C. to ≤250° C.
28. The process according to embodiment 27, wherein the step d) is carried out at a temperature in the range of ≥100° C. to ≤220° C.
29. The process according to embodiment 28, wherein the step d) is carried out at a temperature in the range of ≥150° C. to ≤200° C.
30. The process according to any one of the embodiments 1 to 29, wherein the pH of the step c) and/or step d) is maintained 8.0.
31. The process according to embodiment 30, wherein the pH of the step c) and/or step d) is maintained 7.0.
32. The process according to any one of the embodiments 1 to 31 further comprises a step e) of isolation of an ester or an amide of a compound of formula (I) from the reaction mixture.
33. An ester or an amide of a compound of formula (I) obtained according any one of the embodiments 1 to 32, wherein the ester or the amide of the compound of formula (I) comprises Zr, Zn, Bi, or combination of two or more in free form or a compound form in an amount in the range of 0.0001 to 5.0 wt. % based on total amount of the ester or the amide of the compound of formula (I).
34. The compound according to embodiment 33, wherein the product comprises Zr, Zn, Bi, or combination of two or more in free form or a compound form in an amount in the range of 10 ppm to 5000 ppm.
35. An ester or an amide of a compound of formula (I) comprising Zr, Zn, Bi, or combination of two or more in free form or a compound form in an amount in the range of 0.0001 to 5.0 wt. % based on total amount of the ester or the amide of the compound of formula (O).
36. The compound according to embodiment 35, wherein the product comprises Zr, Zn, Bi, or combination of two or more in free form or a compound form in an amount in the range of 10 ppm to 5000 ppm.
37. A composition comprising an ester or an amide of a compound of formula (I) according to anyone of the embodiments 33 to 36
38. Use of an ester or an amide of a compound of formula (I) according to anyone of the embodiments 33 to 37 as UV light stabilizer.
38. A method of stabilizing a coating/composition against UV light comprising at least the steps of:
   i. proving an ester or an amide of a compound of formula (I) according to any one of the embodiments 33 to 37 or a composition according to embodiment 36.

The presently claimed invention is illustrated in detail by non-restrictive working examples which follow. More particularly, the test methods specified hereinafter are part of the general disclosure of the application and are not restricted to the specific working examples.

### Materials and Methods

Methyl 3-[3-(benzotriazol-2-yl)-5-tert-butyl-4-hydroxy-phenyl]propanoate is available from Alfa Chemistry, USA.

Polyethylene glycol 300 is available under the trade name “Pluriol E 300” from BASF SE, Germany.

Zirconium neodecanoate is available under the trade name “TIB KAT 818” from TIB Chemicals AG, Germany.

Zinc neodecanoate is available under the trade name “TIB KAT 616” from TIB Chemicals AG, Germany.

Bismuth (III)-neodecanoate is available under the trade name “TIB KAT 716” from TIB Chemicals AG, Germany.

A combination of Bismuth (III)-neodecanoate and Zinc neodecanoate is available under the trade name “TIB KAT 718” from TIB Chemicals AG, Germany.

Bi (III) trifluoromethanesulfonic acid is available from Alfa Aesar.

TIn-(II)-2-ethylhexanoate is available from Merck KGaA, Germany.

Polyglycerol-10 is available from Spiga Nord SPA, Italy.

Polyaldo 10-1 S is available from Lonza, Switzerland.

Pentaerythritol and 2-ethylhexanol are available from Aldrich.

Polyethylene glycol 500 monomethyl ether and 1, 1, 1-trimethylolpropane are available from Fluka.

### Example 1: Tinuvin® 1130

Methyl 3-[3-(benzotriazol-2-yl)-5-tert-butyl-4-hydroxy-phenyl]propanoate (195.8 grams, 0.55 mole), toluene (303.6 grams), polyethylene glycol 300 (173.1 grams, 0.60 moles) and zirconium neodecanoate (1.20 grams, 0.003 mole, 0.005 eq) were charged to a reaction flask equipped with an blade impeller, thermometer and attached to a vacuum distillation apparatus equipped with Liebig condenser and receiving flask. Under an argon blanket, the reactor contents were heated to 170° C. and a 10 mbar vacuum was applied. The reaction was monitored by HPLC. After completion of the reaction the desired product was cooled and discharged. The title compound was obtained in a yield of 321 g as a yellow oil.

Examples 2 to 5 conducted same as example 1.

TABLE 1

| | | | | | | residual |
|---|---|---|---|---|---|---|
| | | | | | | starting |
| | | | | | | benzotri- |
| | Catalyst | | Acid | | reaction | azole |
| Ex. | equiv- | chemical | equiv- | chemical | time | compound |
| # | alents | name | alents | name | hh:mm | [%] |
| C1 | 0.01 | tin-(II)- | — | — | 12:20 | 1.5 |
| | | bis(2-ethyl- | | | | |
| | | hexanoate) | | | | |
| 1 | 0.005 | Zr neo- | — | — | 05:30 | 2.5 |
| | | decanoate | | | | |
| 2 | 0.02 | Zr neo- | 0.004 | p-toluenesul- | 02:30 | <0.1 |
| | | decanoate | | fonic acid | | |
| 3 | 0.005 | Zr neo- | 0.005 | p-toluenesul- | 03:30 | 1.4 |
| | | decanoate | | fonic acid | | |
| 4 | 0.005 | Bi neo- | — | — | 03:30 | 0.5 |
| | | decanoate | | | | |
| 5 | 0.007 | Bi neo- | — | p-toluenesul- | 03:00 | 0.2 |
| | | decanoate | | fonic acid | | |

It is evident from table 1 that the catalyst according to presently claimed invention is able to reduce the reaction time compared to tin catalysts. The reaction time can be further reduced by addition of catalytic amount of acid.

Comparative Example C 1: A 100 mL glass flask containing a magnetic stir bar was placed in an agitating heating block. Methyl 3-[3-(benzotriazol-2-yl)-5-tert-butyl-4-hydroxy-phenyl]propanoate (27.0 grams, 0.076 mol), polyethylene glycol 300 (23.9 grams, 0.08 mole) and tin (II) bis(2-ethyl hexanoate) (0.31 grams, 0.00076 mole, 0.01 eq) were transferred into the flask. Under an argon flow, the flask content was heated to 170° C. under vacuum (20 mbar). The reaction was monitored using HPLC. After 12.3 hours the HPLC analysis showed 98.5% conversion of the starting benzotriazole compound to Tinuvine 1130.

The experiments C2, 7-10 were conducted in accordance with C1 with varying amounts of catalysts and the reaction temperatures as indicated in table 2.

TABLE 2

| | | | | | residual |
|---|---|---|---|---|---|
| | | | reaction | reaction | starting |
| | Catalyst | | temperature | time | benzotriazole |
| Ex. # | equivalents | chemical name | ° C. | hh:mm | compound [%] |
| C 1 | 0.01 | tin-(II)-bis(2- | 170 | 12:20 | 1.5 |
| | | ethylhexanoate) | | | |
| C 2 | 0.002 | Tetraisopropyl | 170 | 10:00 | 15 |
| | | orthotitanate | | | |
| 7 | 0.002 | Zr neodecanoate | 170 | 08:00 | 2 |
| 8 | 0.002 | Bi (III) neodecanoate | 170 | 12:00 | 2.6 |
| 9 | 0.002 | Bi (III) neodecanoate | 200 | 05:00 | 5.2 |
| 10 | 0.005 | Bi (III) trifluoro- | 175 | 03:00 | 1.8 |
| | | methanesulfonic acid | | | |
| 11 | 0.01 | zinc neodecanoate | 170 | 04:30 | 0.3 |

### Example 12

A 100 mL glass flask containing a magnetic stir bar was placed in an agitating heating block and 1,1,1,-trimethylolpropane (6.4 grams, 0.047 mole) was transferred into the flask. Under an argon flow, methyl 3-[3-(benzotriazol-2-yl)-5-tert-butyl-4-hydroxy-phenyl]propanoate (12.7 grams, 0.036 mole) and bismuth (III) trifluoromethanesulfonic acid (94.6 milligrams, 0.00014 mole, 0.004 eq) were added. The flask contents were heated up to 175° C. under vacuum (20 mbar) for 3.35 hours. The reaction was monitored using HPLC. HPLC analysis indicated residual amounts of 0.5% of methyl 3-[3-(benzotriazol-2-yl)-5-tert-butyl-4-hydroxy-phenyl]propanoate. The desired product was cooled and discharged. The title compound was obtained in a yield of 16 g as a brownish melt.

Examples 13 to 20 conducted same as example 12.

TABLE 3

| | | | | | | residual | |
|---|---|---|---|---|---|---|---|
| | | | | | | starting | |
| | Alcohol/Amine | | Catalyst | | | benzotriazole | |
| Example | equiv- | chemical | equiv- | chemical | reaction | compound | Yield |
| # | alents | name | alents | name | time hours | [%] | [g] |
| 12 | 1.3 | 1,1,1- | 0.004 | Bi (III) | 3.3 | 0.5 | 19 |
| | | trimethyl- | | trifluorometh- | | | |
| | | olpropane | | anesulfonic | | | |
| | | | | acid | | | |
| 13**⁾ | 0.75 | 1,1,1- | 0.005 | Bi (III) | 7.4 | 4.1 | 19.4 |
| | | trimethyl- | | trifluorometh- | | | |
| | | olpropane | | anesulfonic | | | |
| | | | | acid | | | |
| 14 | 2.2 | 1,1,1- | 0.004 | Bi (III) | 3.0 | 0.5 | 19 |
| | | trimethyl- | | trifluorometh- | | | |
| | | olpropane | | anesulfonic | | | |
| | | | | acid | | | |
| 15 | 2.4 | penta- | 0.004 | Bi (III) | 3.0 | 2.2 | 19.9 |
| | | erythritol | | trifluorometh- | | | |
| | | | | anesulfonic | | | |
| | | | | acid | | | |
| 16 | 1.3 | polyethylene | 0.006 | TIB KAT 818 | 11.3 | 1.5 | 37.5 |
| | | glycol 500 | | | | | |
| | | monomethyl | | | | | |
| | | ether | | | | | |
| 17*^{,}** | 0.2 | polygly- | 0.01 | TIB KAT 716 | 10.5 | 2.5 | 22.9 |
| | | cerol-10 | | | | | |
| 18*^{,}** | 0.2 | polygly- | 0.01 | TIB KAT 616 | 16.0 | 3.1 | 20.8 |
| | | cerol-10 | | | | | |
| 19*^{,}** | 0.2 | Polyaldo | 0.01 | TIB KAT 818 | 28.0 | 2.8 | 25 |
| | | 10-1 S | | | | | |
| 20*^{,}** | 0.3 | Triglycerol | 0.01 | TIB KAT 616 | 4.5 | 0.9 | 19.2 |

*reactions were carried out at 200° C.
**reactions were carried out with 0.05 mole of methyl 3-[3-(benzotriazol-2-yl)-5-tert-butyl-4-hydroxy-phenyl]propanoate.

### Example 24: Tinuvin® 1130

Methyl 3-[3-(benzotriazol-2-yl)-5-tert-butyl-4-hydroxy-phenyl]propanoate (179.1 grams), toluene (200.4 grams), polyethylene glycol 300 (164.5 grams) and TIB Kat 718 (1.82 grams) were charged to a reaction flask equipped with an blade impeller, thermometer and attached to a vacuum distillation apparatus equipped with Liebig condenser and receiving flask. Under an argon blanket, the reactor contents were heated to 170° C. and a 20 mbar vacuum was applied. The reaction was monitored by HPLC. After completion of the reaction the desired product was cooled and discharged. The title compound was obtained in a yield of 266.6 g as a yellow oil.

### Example 25: [2-[3-[3-(benzotriazol-2-yl)-5-tert-butyl-4-hydroxy-phenyl]propanoyloxy methyl]-3-hydroxy-2-(hydroxymethyl)propyl]3-[3-(benzotriazol-2-yl)-5-tert-butyl-4-hydroxy phenyl]propanoate

A 100 mL glass flask containing a magnetic stir bar was placed in an agitating heating block and pentaerythritol (3.1 grams, 0.022 mole) was transferred into the flask. Under an argon flow, 3-[3-(benzotriazol-2-yl)-5-tert-butyl-4-hydroxy-phenyl]propanoic acid (16.9 grams, 0.050 mole) and bismuth (III) trifluoromethanesulfonic acid (132.4 milligrams, 0.0003 mole, 0.006 eq) were added. The flask contents were heated up to 175-178° C. under vacuum (20 mbar) for 5 hours. The reaction was monitored using HPLC. After 5 hours HPLC analysis indicated residual amounts of 1.0% of 3-[3-(benzotriazol-2-yl)-5-tert-butyl-4-hydroxy-phenyl]propanoic acid. The desired product was cooled and discharged. The title compound was obtained in a yield of 18.4 g as a brownish melt.

### Example 26

The experiment 26 was conducted in accordance with example 25 using 3-[3-(benzotriazol-2-yl)-5-tert-butyl-4-hydroxy-phenyl]propanoic acid (16.9 grams, 0.050 mole), 0.2 equivalents of polyglycerol-10 (7.6 grams) and 0.01 equivalents of TIB KAT 716 at a reaction temperature of 2000 C. After 4.5 hours HPLC analysis indicated residual amounts of 0.4% of 3-[3-(benzotriazol-2-yl)-5-tert-butyl-4-hydroxy-phenyl]propanoic acid yielding 23.3 grams of a brownish melt.

### Example 27: Tinuvin® 1130

Methyl 3-[3-(benzotriazol-2-yl)-5-tert-butyl-4-hydroxy-phenyl]propanoate (195.8 grams, 0.55 mole), toluene (304 grams), polyethylene glycol 300 (179 grams, 0.60 moles), Bi (III) neodecanoate (TIB KAT 716, 2.8 grams, 0.004 mole) and p-toluene sulfonic acid (0.53 g, 0.003 mole) were charged to a reaction flask equipped with an blade impeller, thermometer and attached to a vacuum distillation apparatus equipped with Liebig condenser and receiving flask. Under an argon blanket, the reactor contents were heated to 170° C. and a 10 mbar vacuum was applied. After 2.5 hours a TLC analysis indicated residual amounts of less than 2% of the starting benzotriazole methyl 3-[3-(benzotriazol-2-yl)-5-tert-butyl-4-hydroxy-phenyl]propanoate. The desired product was then filtrated yielding 338 g of a yellow oil. The product was analyzed by ICP-AES (Inductively Coupled Plasma Atomic Emission Spectrometry) for Bi and was found to contain 1060 ppm of Bi.

Example 28: Tinuvin® 1130 with Zirconium neodecanoate and carbonic and sulfonic acids A 100 mL glass flask containing a magnetic stir bar was placed in an agitating heating block. Polyethylene glycol 300 (15.7 grams, 0.05 mole), methyl 3-[3-(benzotriazol-2-yl)-5-tert-butyl-4-hydroxy-phenyl]propanoate (17.6 grams, 0.05 mol) and zirconium neodecanoate (TIB KAT 818, 0.185 grams, 0.0004 mole, 0.008 eq) and benzoic acid (0.0304 grams, 0.0002 mole) were transferred into the flask. Under an argon flow, the flask content was heated to 175° C. under vacuum (20 mbar). The reaction was monitored using HPLC. After 11 hours the HPLC analysis showed 96.7% conversion of the starting benzotriazole compound to Tinuvin® 1130 yielding 31.3 g of an orange, clear melt.

The experiments 29-31 were conducted in accordance with example 28 with different and varying amounts of catalysts and carbonic or sulfonic acids as indicated in table 4.

TABLE 4

| | | | | | | residual | |
|---|---|---|---|---|---|---|---|
| | | | | | | starting | |
| | Catalyst | | Acid | | | benzotriazole | |
| | equiv- | chemical | equiv- | chemical | reaction | compound | Yield |
| Ex # | alents | name | alents | name | time hours | [%] | [g] |
| 28 | 0.00855 | TIB KAT | 0.005 | benzoic acid | 11.0 | 3.1 | 31.3 |
| 29 | 0.00827 | 818 | 0.005 | 3-methoxy- | 15.0 | 0.65 | 32.2 |
| | | | | benzoic acid | | | |
| 30 | 0.00745 | | 0.005 | 4-dodecyl- | 7.6 | 3.6 | 23.3 |
| | | | | benzene | | | |
| | | | | sulfonic acid | | | |
| 31 | 0.005 | | 0.01 | camphor-10- | 7.6 | 0.8 | 28.5 |
| | | | | sulfonic acid | | | |

Example 32: Octyl 3-[3-(benzotriazol-2-yl)-5-tert-butyl-4-hydroxy-phenyl]propanoate Methyl 3-[3-(benzotriazol-2-yl)-5-tert-butyl-4-hydroxy-phenyl]propanoate (58.7 grams, 0.17 mole), isooctanol (43.3 grams, 0.33 mol, from Exxon) and TIB KAT 818 (0.734 grams, 1.66 mmole) were charged to a reaction flask equipped with a blade impeller. Under an argon blanket, the reactor contents were heated to 155° C. and a 220 mbar vacuum was applied. After holding at 155° C. for 4 hours, the excess of isooctanol was distilled off by reducing the pressure to 1 mbar. The desired product was cooled and discharged. The title compound was obtained as a yellow liquid (79.3 g).

The product obtained were analyzed for the residual metal using ICP-AES (Inductively Coupled Plasma Atomic Emission Spectrometry). The residual metal in the examples as shown in table 5.

TABLE 5

| Example # | Metal | Amount of residual metal in ppm |
|---|---|---|
| 11 | Zn | 930 |
| 27 | Bi | 1060 |

### Example 33: Tinuvin® 1130 with Bi-Neodecanoate and Carbonic Acids

A 100 mL glass flask containing a magnetic stir bar was placed in an agitating heating block. Polyethylene glycol 300 (15.7 grams, 0.05 mole), methyl 3-[3-(benzotriazol-2-yl)-5-tert-butyl-4-hydroxy-phenyl]propanoate (17.6 grams, 0.05 mol), bismuth neodecanoate (TIB KAT 716, 0.323 grams, 0.00025 mole, 0.005 eq) and 2-phenylpropionic acid (0.04 grams, 0.00025 mole) were transferred into the flask. Under an argon flow, the flask content was heated to 175° C. under vacuum (20 mbar). The reaction was monitored using HPLC. After 4.5 hours the HPLC analysis showed complete conversion of the starting benzotriazole compound to Tinuvin® 1130 yielding 34.3 g of a brownish melt.

The experiment 34 was conducted in accordance with example 33 with different and varying amount of catalysts and carbonic acid as indicated in table 6.

TABLE 6

| | | | | | | residual | |
|---|---|---|---|---|---|---|---|
| | | | | | | starting | |
| | Catalyst | | Acid | | | benzotriazole | |
| | equiv- | chemical | equiv- | chemical | reaction | compound | Yield |
| Ex # | alents | name | alents | name | time hours | [%] | [g] |
| 33 | 0.005 | TIB KAT | 0.006 | 2-phenyl- | 4.5 | 0.0 | 31.3 |
| | | 716 | | propionic acid | | | |
| 34 | 0.003 | | 0.001 | 2-methyl-2- | 8.3 | 0.6 | 31.5 |
| | | | | phenyl- | | | |
| | | | | propoinic acid | | | |

TABLE 6

| Formulation based on Acronal LR 9014 | Weight % |
|---|---|
| AMP 90 | 23 |
| Hydropalat WE 3240 | 0.1 |
| Foamex 810 | 0.1 |
| Propylenglycol | 0.4 |
| Solvenon DPM | 2.00 |
| Acronal LR 9014 | 2.00 |
| Rheovis PU 1214 | 70.00 |
| Rheovis PE 1330 | 0.7 |
| water | 0.5 |
| % solid content | 34.80 |

Each coating formulation A was stabilized with 1.0 or 3.0 active % based on resin solids of Tinuvin® 1130 (UV absorber, BASF SE), Tinuvin® 9945-DW (N) or inventive examples samples.

Each coating formulation further contained 1,0 active % based on resin solids of Tinuvin® 123DW (N) (HALS, BASF SE).

The clear coat formulations were subsequently applied by brush onto a wood substrate (thickness: two layers à 100 g/m² resulting after drying at room temperature in a final dry film thickness of ˜40 μm). The clear coat formulations were subsequently applied by draw-down onto a glass plate (thickness: one layer b 200 g/m² resulting after drying at room temperature in a final dry film thickness of ˜40 μm).

### Solubility Tests

The solubility of above-described coating formulation containing one of the compounds obtained in examples 3, 11 and 27 in the amount given above was tested. The solubility was visually assessed qualitatively by presence of defects in the dried paint. Results are given in the following table:

TABLE 7

| Raw material | Presence of defects |
|---|---|
| 1% Tinuvin ® 1130 | no |
| 3% Tinuvin ® 1130 | no |
| 1% Tinuvin ® 9945-DW (N) | no |
| 3% Tinuvin ® 9945-DW (N) | no |
| 1% example 3 | no |
| 1% example 11 | no |
| 1% example 27 | no |

### Compatibility Tests

The compatibility of above described coating formulation containing one of the compounds obtained in examples 3, 11 and 27 in the amount given above was tested. For the assessment of the compatibility, the haze, the 20° gloss, the pendulum hardness (PH) and the discoloration (ΔE*) vs. non-stabilized formulation were measured (formulations were applied onto a white/black coil-coating panel to a final DFT of 35-40 μm). Results are given in the following table:

TABLE 8
Tested in L21039 series:

| Raw material | Haze | Gloss | PH | ΔE* |
|---|---|---|---|---|
| Non-stabilized | 36.0 | 76.2 | 26 | — |
| 1% Tinuvin ® 1130 | 33.0 | 76.6 | 21 | 0.2 |
| 3% Tinuvin ® 1130 | 34.0 | 77.1 | 18 | 0.4 |
| 1% example 3 | 35.0 | 74.3 | 22 | 0.2 |
| 3% example 3 | 37.0 | 77.0 | 19 | 0.5 |
| 1% example 11 | 33.0 | 76.1 | 23 | 0.1 |
| 3% example 11 | 38.0 | 76.7 | 20 | 0.4 |
| 1% example 27 | 35.0 | 76.9 | 22 | 0.3 |
| 3% example 27 | 34.0 | 76.6 | 20 | 0.4 |

### Accelerated Weathering Tests

Coatings comprising the compounds (in addition to 1,0% active on binder solid content Tinuvin® 123-DW (N)) were tested under artificial weathering cycles to evaluate their performances, as indicated by the discoloration (ΔE*) after a given duration of the exposure. The coatings were applied onto a glass plate to a DFT of around 40 μm.

TABLE 9
Accelerated weathering results:

| | Δ E* after . . . h Xe-WOM | | | | |
|---|---|---|---|---|---|
| | (ISO 16474-2) exposure: | | | | |
| | 960 | 1920 | 2880 | 3840 | 4800 |
| Non-stabilized | 0.1 | 0.2 | 0.3 | 0.1 | 0.3 |
| 3% Tinuvin ® 1130 + 1% Tinuvin ® | 0.1 | 0.2 | 0.2 | 0.1 | 0.1 |
| 123-DW (N) | | | | | |
| 3% example 3 + 1% Tinuvin ® | 0.0 | 0.1 | 0.2 | 0.1 | 0.1 |
| 123-DW (N) | | | | | |
| 3% example 5 + 1% Tinuvin ® | 0.1 | 0.1 | 0.2 | 0.0 | 0.1 |
| 123-DW (N) | | | | | |
| 3% example 11 + 1% Tinuvin ® | 0.0 | 0.1 | 0.2 | 0.1 | 0.1 |
| 123-DW (N) | | | | | |

It is evident that the product of presently claimed invention displayed similar/better UV stabilizing property compared to the comparative.

1.-15. (canceled)

16. A process for obtaining an ester or an amide of a compound of formula (I) comprising at least the step of:
a) providing at least one compound of formula (I), 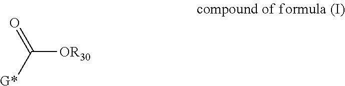
wherein
G* is selected from formula (A), or formula (B) 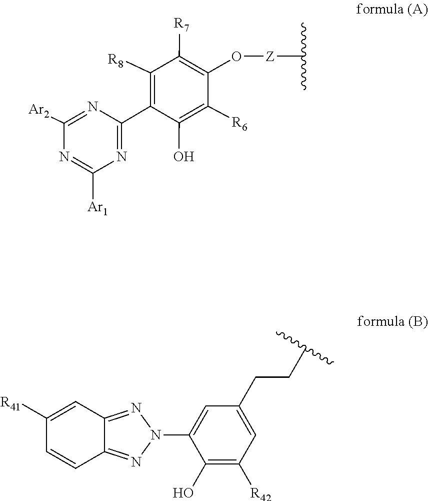
wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C), or a moiety of formula (M), 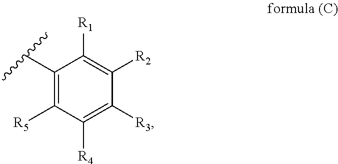
wherein R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, OR,
R selected from hydrogen, substituted or unsubstituted, linear or branched C₁-C₂₄ alkyl, 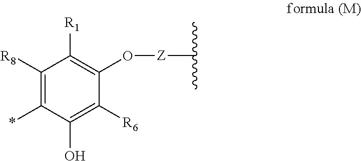
wherein the dotted line is a single bond between formula (M) and the triazinyl ring of formula (A), Z is as defined as above,
R₆, R₇ and R₈ are independently of each other selected from hydrogen, substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
b) providing at least one alcohol (D) or an amine (E) to obtain a mixture,
c) optionally providing at least one organic acid (F) to mixture obtained in step b) to obtain a reaction mixture, and
d) reacting the mixture obtained in step b) or step c) in the presence of at least one catalyst selected from Zr compound, Bi compound, Zn compound, or a combination of two or more thereof.

17. The process according to claim 16, wherein the Zr compounds are selected from ZrO₂, Zirconium halide, Zr(O(O)CR₆₁)₂, Zr(═O)(O(O)CR₆₁)₂, Zr(O(O)CR₆₁)₄, Zr(O(O)CR₆₂C(O)O), or Zr(O(O)CR₆₂C(O)O)₂, Zr(O(O)₂SR₆₁)₂, Zr(O(O)₂SR₆₁)₄, Zr(O(O)₂SR₆₂S(O)₂₀), or Zr(O(O)₂SR₆₂S(O)₂₀)₂, wherein R₆₁ is selected form substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl; and R₆₂ is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene.

18. The process according to claim 16, wherein the Zn compound is selected from Zinc oxide, Zinc halide, Zn(O(O)CR₆₁)₂, or Zn(O(O)CR₆₂C(O)O), wherein R₆₁ is selected form substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl; and R₆₂ is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene.

19. The process according to claim 16, wherein the Bi compound is selected from Bismuth halide, Bismuth oxide, Bi(R₆₃)₂(O(O)CR₆₁), Bi(R₆₃)(O(O)CR₆₂C(O)O), or Bi(O(O)CR₆₁)₃, Bi(OR₆₃)₃, Bi(R₆₃)₂(O(O)₂SR₆₁), Bi(R₆₃)(O(O)₂SR₆₂S(O)₂₀), or Bi(O(O)₂SR₆₁)₃, wherein R₆₁ is selected form substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl; and R₆₂ is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene, and R₆₃ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl.

20. The process according to claim 16, wherein the at least one organic acid (F) is different from the acid of compound of formula (I).

21. The process according to claim 16, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tertbutyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxoethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxoethoxy)phenyl]-6-(4-methoxyphenyl)-1,3,5-triazin-2-yl]phenoxy]propanoate, or a combination of two or more thereof.

22. The process according to claim 16, wherein the alcohol (D) is a monohydric or a polyhydric alcohol.

23. The process according to claim 16, wherein the alcohol (D) is selected from pentaerythritol, trimethylolpropane, ethylene glycol, substituted or unsubstituted C₁₋₃₀ alkanol having one or more hydroxy functional groups, substituted or unsubstituted C₃₋₃₀ alkenol having one or more hydroxy functional groups, substituted or unsubstituted C₃₋₃₀ heteroalkanol having one or more hydroxy functional groups, substituted or unsubstituted C₄₋₃₀ hetero alkenol having one or more hydroxy functional groups, substituted or unsubstituted aromatic phenol having one or more hydroxy functional groups, substituted or unsubstituted aralkyl having one or more hydroxy functional groups, substituted or unsubstituted cyclic alcohol having one or more hydroxy functional groups, polyalkyleneoxide having one or more hydroxy functional groups, substituted or unsubstituted polyglycerols, or a combination of two or more thereof.

24. The process according to claim 16, wherein the mole ratio of compound of formula (I) to the total amount of alcohol (D) or amine (E) is in the range of 50:0.1 to 0.1:50.

25. The process according to claim 16, wherein the catalyst in the reaction is present in an amount in the range of 0.0001 to 30 wt. % based on total weight of compounds of formula (I).

26. An ester or an amide of a compound of formula (I) obtained according claim 16, wherein the ester or the amide of the compound of formula (I) comprises Zr, Zn, Bi, or combination of two or more in free form or a compound form in an amount in the range of 0.0001 to 5.0 wt. % based on total amount of the ester or the amide of a compound of formula (I).

27. An ester or amide of compound of formula (I) comprises Zr, Zn, Bi, or combination of two or more in free form or a compound form in an amount in the range of 0.0001 to 5.0 wt. % based on total amount of the compound of formula (I).

28. A composition comprising the ester or amide of a compound of formula (I) according to claim 27.

29. Use of the ester or amide of a compound of formula (I) according to claim 27 as UV light stabilizer.

30. A method of stabilizing a coating/composition against UV light comprising at least the steps of:
a. providing the ester or amide of a compound of formula (I) according to claim 27.
